(19) United States
(12) Patent Application Publication
(10) Pub. No.: US 2026/0262949 A1
(43) Pub. Date: Sep. 10, 2026

(54) TEMPERATURE SAFETY SYSTEMS AND METHODS

(71) Applicant: Kandu Health, Inc., Campbell, CA (US)

(72) Inventors: Lindsay Howard, San Jose, CA (US); Michael Strasser, Corte Madera, CA (US); Benjamin Micah Berkowitz, San Carlos, CA (US)

(21) Appl. No.: 19/480,007

(22) PCT Filed: May 2, 2024
(86) PCT No.: PCT/US2024/027425

§ 371 Date: Oct. 29, 2025

Related U.S. Application Data
(60) Provisional application No. 63/464,345, filed on May 5, 2023.

(51) Int. Cl. A61B 5/02; A61B 5/00; A61B 5/01; G01K 3/00

(52) U.S. Cl. CPC A61B 5/02007; A61B 5/01; A61B 5/681; G01K 3/005; A61B 2560/0214; A61B 2562/0271

(57) ABSTRACT

A temperature safety system for a wearable device is disclosed. The temperature safety system can comprise a power source to provide power to a heating source, a first switch to receive a first input from a control system; and a second switch to receive a second input from a hardware electronic circuit. The heating source can receive power from the power source in response to activation of both the first switch and the second switch. A control switch can be disposed between the power source and the heating source to receive an input related to an input from at least one of the first switch and the second switch. The control system can comprise a first temperature sensor to measure a first temperature reading corresponding to a temperature of a skin surface receiving a stimulus from the heating source.

### CROSS-REFERENCE TO RELATED APPLICATIONS

This application claims the priority benefit of U.S. Provisional Patent Application No. 63/464,345, filed May 5, 2023, the contents of which are herein incorporated by reference in their entireties.

### TECHNICAL FIELD

This disclosure relates generally to the field of non-invasive detection and estimation of physiological signals and, more specifically, to the prevention of burns during the detection of physiological signals.

### BACKGROUND

Stroke is the third most common cause of death in the United States and the most disabling neurologic disorder. Approximately 800,000 patients suffer from stroke annually, and there are about 6 to 8 million stroke survivors. Stroke is a medical emergency characterized by the acute onset of a neurological deficit that persists for at least 24 hours, reflecting focal involvement of the central nervous system, and is the result of a disturbance of the cerebral circulation. Its incidence increases with age. Risk factors for stroke include systolic or diastolic hypertension, hypercholesterolemia, cigarette smoking, heavy alcohol consumption, diabetes, and oral contraceptive use.

Hemorrhagic stroke accounts for about 13% of the annual stroke population. Hemorrhagic stroke often occurs due to rupture of an aneurysm or arteriovenous malformation bleeding into the brain tissue, resulting in cerebral infarction. The remaining 87% of the stroke population are ischemic strokes and are caused by occluded vessels that deprive the brain of oxygen-carrying blood. Ischemic strokes are often caused by emboli or pieces of thrombotic tissue that have dislodged from other body sites or from the cerebral vessels themselves to occlude in the narrow cerebral arteries more distally. When a patient presents with neurological symptoms and signs which resolve completely within 1 hour, the term transient ischemic attack (TIA) is used. Etiologically, TIA and stroke share the same pathophysiologic mechanisms and thus represent a continuum based on persistence of symptoms and extent of ischemic insult.

Notwithstanding the foregoing, once the patient is discharged from the hospital, the patient's road to recovery is long and arduous, especially since existing therapies are incomplete at treating or reversing the effects of the stroke. Many disabilities or effects of the stroke may present early or may not present until days or weeks or months later. As such, patients and their caregivers require many resources, tools, and support in adapting to their current state, recovering after the stroke event, and connecting with their doctor, care network, and other survivors. Accordingly, there exists a need for improved stroke care management after a stroke event.

### SUMMARY

Described herein are systems, devices, and methods for monitoring and/or characterizing vasomotor activity and/or responses.

Disclosed herein is a temperature safety system for a wearable device, the temperature safety system including: a power source configured to provide power to a heating source; a first switch configured to receive a first input from a control system; and a second switch configured to receive a second input from a hardware electronic circuit, wherein the heating source is configured to receive power from the power source in response to activation of both the first switch and the second switch.

In some implementations, the system includes a control switch disposed between the power source and the heating source, the control switch configured to receive an input related to an input from at least one of the first switch and the second switch. In some implementations, control system includes a first temperature sensor configured to measure a first temperature reading, the first temperature reading corresponding to a temperature of a skin surface receiving a stimulus from the heating source. In some implementations, the control system includes one or more processors in communication with the first temperature sensor, the one or more processors configured to receive the first temperature reading from the first temperature sensor.

In some implementations, the one or more processors are configured to: receive signals of the first temperature reading from the first temperature sensor; process the signals of the first temperature reading to determine whether the signals of the first temperature reading are less than a first temperature threshold; and output a first activation signal or a first cutoff signal at least based on the determination as the first input to the first switch. In some implementations, the first temperature threshold is 44° C. In some implementations, the first temperature threshold of 44° C. is a maximum temperature threshold. In some implementations, the first temperature threshold is 42° C. In some implementations, the hardware circuit includes a second temperature sensor in communication with the heating source, the second temperature sensor configured to measure a second temperature reading, the second temperature reading corresponding to a temperature of the heating source.

In some implementations, the system includes comparators to compare the second temperature reading to a second threshold value. In some implementations, the techniques described herein relate to a system, wherein the hardware electronic circuit is configured to: receive signals of the second temperature reading from the second temperature sensor; process the signals of the second temperature reading using the comparators to determine whether the signals of the second temperature reading are less than the second temperature threshold; and output a second activation signal or a second cutoff signal at least based on the determination as the second input to the second switch.

In some implementations, the system includes one or more control modules in communication with at least one of the first switch and the second switch, the one or more control modules configured to receive an input from at least one of the first switch and the second switch. In some implementations, the one or more control modules include field effect transistors.

Disclosed herein is a wearable system having a temperature safety system to prevent thermal injury to a person, the system including: a wearable device configured to be in contact with a skin surface of a person, said wearable device including: a stimulus source in communication with the skin surface, wherein the stimulus source is configured to heat the skin surface to a target temperature, wherein the target temperature is a baseline skin temperature of the skin surface plus about 1 degree to about 20 degrees; a first temperature sensor configured to measure a first temperature reading, the first temperature reading including a temperature of the skin surface; one or more processors in communication with the first temperature sensor, the one or more processors configured to receive the first temperature reading from the first temperature sensor; a circuit including a second temperature sensor in communication with the stimulus source, the second temperature sensor configured to measure a second temperature reading, the second temperature reading including a temperature of the stimulus source; and a power source in electrical communication with the stimulus source and configured to provide power to the stimulus source; wherein the one or more processors are configured to: receive signals of the first temperature reading from the first temperature sensor; process the signals of the first temperature reading to determine whether the signals of the first temperature reading are less than a first temperature threshold; and output a signal to the power source and the stimulus source to initiate a stimulus cycle, the stimulus cycle including heating the skin surface to the target temperature; and wherein the second temperature sensor is configured to: receive signals of the second temperature reading from the second temperature sensor; process the signals of the second temperature reading to determine whether the signals of the second temperature reading are less than a second temperature threshold; and output a signal to the power source and the stimulus source to initiate a stimulus cycle, the stimulus cycle including heating the skin surface to the target temperature.

In some implementations, one or more processors are configured to monitor the first temperature reading during the stimulus cycle. In some implementations, the first temperature threshold is 44° C. In some implementations, the first temperature threshold of 44° C. is a maximum temperature threshold. In some implementations, the first temperature threshold is 42° C. In some implementations, the second temperature sensor is configured to monitor the second temperature reading during the stimulus cycle. In some implementations, the second temperature threshold is 48° C. In some implementations, the first temperature threshold of 48° C. is a maximum temperature threshold.

In some implementations, the first temperature sensor includes a thermocouple, a resistance temperature detector, a thermistor, or an infrared temperature sensor. In some implementations, the second temperature sensor is positioned at least near a center of the stimulus source. In some implementations, the second temperature sensor includes a thermocouple, a resistance temperature detector, a thermistor, or an infrared temperature sensor.

In some implementations, the power source is a battery or a port for connecting the wearable device to a power supply. In some implementations, the stimulus source cycles on and off until the target temperature is reached. In some implementations, the system includes one or more control modules in communication with at least one of the one or more processors and the circuit, the one or more control modules configured to receive an input from at least one of the one or more processors and the circuit. In some implementations, the one or more control modules include field effect transistors. In some implementations, the one or more control modules disconnect the stimulus source from the power source based on the input from at least one of the one or more processors and the circuit. In some implementations, the one or more control modules are positioned in series. In some implementations, the one or more control modules are positioned in parallel. In some implementations, the system includes a remote computing device communicative coupled to the one or more processors.

In some implementations, the remote computing device includes one of: a laptop, cellular device, a workstation, a server, a desktop computer, a personal digital assistant, a second wearable system or device, or a netbook. In some implementations, the system includes at least one tensionable band coupled to the person.

Disclosed herein is a safety system of a wearable system to manage a stimulus cycle to prevent a burning of a person, the safety system including: one or more processors in electrical communication with a first temperature sensor, the first temperature sensor configured to measure a first temperature of a skin surface site being heated by a stimulus source powered by a power source; a circuit including a second temperature sensor in communication with the stimulus source, the second temperature sensor configured to measure a second temperature reading, the second temperature reading including a temperature reading of the stimulus source; and wherein the one or more processors are configured to: receive signals of a first temperature reading from the first temperature sensor; process the signals of the first temperature reading to determine whether the signals of the first temperature reading are less than a first temperature threshold; and output a signal to the power source and the stimulus source to initiate a stimulus cycle, the stimulus cycle including heating the skin surface to a target temperature; wherein the second temperature sensor is configured to: receive signals of the second temperature reading from the second temperature sensor; process the signals of the second temperature reading to determine whether the signals of the second temperature reading are less than a second temperature threshold; and output a signal to the power source and the stimulus source to initiate a stimulus cycle, the stimulus cycle includes heating the skin surface to the target temperature.

In some implementations, the system includes one or more control modules positioned between the power source and the stimulus source and in communication with at least one of the one or more processors and the circuit, the one or more control modules configured to receive an input from at least one of the one or more processors and the circuit, wherein the control modules are configured to disconnect the power source from the stimulus source based on the signal from either the first temperature reading or the second temperature reading. In some implementations, the one or more control modules disconnect the power source from the stimulus source based on the signal from both the first temperature reading and the second temperature reading. In some implementations, the one or more control modules disconnect the power source from the stimulus source based on the first temperature reading exceeding the first temperature threshold. In some implementations, the one or more control modules disconnect the power source from the stimulus source based on the second temperature reading exceeding the second temperature threshold. In some implementations, the one or more control modules include field effect transistors. In some implementations, the first temperature threshold is 44° C. In some implementations, the first temperature threshold of 44° C. is a maximum temperature threshold. In some implementations, the first temperature threshold is 42° C. In some implementations, the second temperature threshold is 48° C. In some implementations, the first temperature threshold of 48° C. is a maximum temperature threshold. In some implementations, the power source is a battery or a port for connecting to a power supply. In some implementations, the system includes processing the signals of the first temperature reading to determine whether the wearable system is in contact with the skin surface site of the person; and output a signal to the stimulus source to terminate the stimulus cycle based on the skin temperature reading.

Disclosed herein is a method of preventing harm to a user from a wearable device, the method including: determining a first temperature based at least on a first temperature reading from a first temperature sensor, the first temperature sensor in communication with one or more processors, wherein the first temperature reading includes a temperature of the user; determining a second temperature based at least on a second temperature reading from a second temperature sensor monitoring a stimulus source, wherein the second temperature includes a temperature reading of the stimulus source; receiving a first signal of the first temperature reading from the first temperature sensor; receiving a second signal of the second temperature reading from the second temperature sensor, processing the first signal of the first temperature to determine whether the first temperature reading are less than a first temperature threshold; processing the second signal of the second temperature to determine whether the second temperature reading are less than a second temperature threshold; outputting a signal to a power source and the stimulus source to initiate a stimulus cycle based at least on the first temperature being less than a first threshold temperature and the second temperature being less than a second temperature threshold, the stimulus cycle including heating a skin surface site to a target temperature sensor.

In some implementations, the first temperature further includes a temperature reading of a skin surface site receiving a stimulus form the stimulus source. In some implementations, the first temperature sensor includes a thermocouple, a resistance temperature detector, a thermistor, or an infrared temperature sensor. In some implementations, the second temperature sensor is positioned near a center of the stimulus source. In some implementations, the second temperature sensor includes a thermocouple, a resistance temperature detector, a thermistor, or an infrared temperature sensor.

In some implementations, the power source is a battery or a port for connecting the wearable device to a power supply. In some implementations, the method includes cycling the stimulus source cycles on and off until the target temperature is reached. In some implementations, the method includes transmitting the processed first signal and the second signal to one or more control modules in communication with at least one of the first temperature sensor and the second temperature sensor. In some implementations, the one or more control modules include field effect transistors. In some implementations, the method includes disconnecting the stimulus source from the power source at least based on the processed first signal and the second signal. In some implementations, the method includes processing the method on a remote computing device communicative coupled to the one or more processors. In some implementations, the remote computing device includes one of: a laptop, cellular device, a workstation, a server, a desktop computer, a personal digital assistant, a second wearable system or device, or a netbook.

Disclosed herein is a wearable device for determining one or more physiological parameters indicative of multivariate stroke, the wearable device including: a body portion including: a housing including a first surface, a second surface opposite said first surface, a plurality of side surfaces extending between the first and second surfaces, an interior, and a battery positioned within the interior; a first band connected to a first side of the plurality of sides of the housing, said first band configured to secure the body portion to a user by wrapping around at least a portion of the user's body, wherein said second surface of said housing is configured to contact skin of the user's body when the first band secures the body portion to the user; and a connector port configured to receive a cable along an insertion axis for charging said battery, said connector port is positioned at a location on said first side of the housing where the insertion axis at least partially overlaps a width of the first band.

In some implementations, said first band is connected to said first side at a first location; and said connector port is positioned at a second location on said first side that is between said first location and said second surface of the housing. In some implementations, said first and second location are substantially vertically aligned with one another. In some implementations, the cable is at least one of a power cable and a data transfer cable. In some implementations, when the cable is connected to the connector port and said second surface of said housing is positioned to contact said skin, the cable providers a physical interference that inhibits said first band from wrapping around said at least the portion of the user's body.

In some implementations, the wearable device includes a second band connected to a second side of the plurality of sides of the housing, said second band configured to secure the body portion to the user by wrapping around at least a portion of the user's body and buckling to the first band. In some implementations, the wearable device includes a second connector port configured to removably connect to a cable to allow charging of said battery, said second connector port positioned at a location on said second side of the housing such that the second connector port intersects at least a portion of the second band. In some implementations, connecting the cable to the second connector port and said second surface of said housing is positioned to contact said skin, the cable provides a physical interference that inhibits said second band from wrapping around said at least a portion of the user's body.

In some implementations, the battery is configured to be charged when the cable is connected to the connector port concurrently with the second cable connected to the second connector port. In some implementations, the wearable device includes a second battery disposed in the interior, wherein the second battery is configured to be charged when the second cable is connected to the second connector port. In some implementations, the wearable device includes a wireless magnetic sensor disposed on the second surface and configured to magnetically attach to a wireless magnetic pad, wherein the wireless magnetic sensor determines whether the body portion is located on the wireless magnetic pad before the device is charged. In some implementations, the wireless magnetic pad is configured to charge the battery.

Disclosed herein is a wearable device configured to be secured to a user's body, the device including: a body portion including a housing, an interior within the housing, and a battery positioned within the interior; a band connected to a first portion of the housing, said band configured to secure the body portion to the user by wrapping around at least a portion of the user's body; and a connector port configured to removably connect to a cable to allow charging of said battery, said connector port positioned at a second portion of the housing relative to said first portion of the housing such that said connector port positioned at a location on said first side of the housing such that the connector port intersects at least a portion of the band.

In some implementations, said band is connected to said first portion at a first location; and said connector port is positioned at a second location on said housing that is between said first location and a surface of the housing configured to contact skin of the user's body when the first band secures the body portion to the user. In some implementations, said first and second location are substantially vertically aligned with one another. In some implementations, said band is connected to said first portion at a first location; and said connector port is positioned at a surface of the housing configured to contact skin of the user's body when the first band secures the body portion to the user.

In some implementations, the cable is at least one of a power cable and a data transfer cable. In some implementations, when the cable is connected to the connector port and the body portion is positioned adjacent the user's body, the cable providers a physical interference that inhibits said band from wrapping around said at least the portion of the user's body.

In some implementations, the device includes a second band connected to a third portion of the housing, said second band configured to secure the body portion to the user by wrapping around at least a portion of the user's body and buckling to the first band. In some implementations, the device includes a second connector port configured to removably connect to a cable to allow charging of said battery, said second connector port positioned at a fourth portion of the housing relative to said third portion of the housing such that, when the cable is connected to the connector port and the body portion is positioned adjacent the user's body, the cable provides a physical interference that inhibits said second band from wrapping around said at least the portion of the user's body. In some implementations, the battery is configured to be charged when the cable is connected to the port concurrently with the second cable connected to the second connector port.

In some implementations, the device includes a second battery disposed in the interior, wherein the second battery is configured to be charged when the second cable is connected to the second connector port. In some implementations, the device includes a wireless magnetic sensor disposed on the housing and configured to magnetically attach to a wireless pad, wherein the wireless magnetic sensor determines whether the body portion is located on the wireless magnetic pad before the battery is charged. In some implementations, the wireless magnetic pad is configured to charge the power source.

Disclosed herein is a charging system for a wearable device, the charging system including: a body having a first surface opposite a second surface in contact with a skin surface of a person, the first surface and second surface defining an interior space, wherein a power source is disposed in the interior space; a plurality of sides disposed between the first surface and the second surface; a first band coupled to a first side of the plurality of sides, the first band including a first edge and a second edge opposite of the first edge defining a width of the first band; a second band coupled to a second side of the plurality of sides opposite of the first band, the second band including a first edge and a second edge opposite of the first edge defining a width of the second band wherein, the first band and the second band are configured to wrap around a portion of a user; a connector port disposed on a side surface of the plurality of sides configured to be removably connected to a cable providing power to the power source, the connector port positioned between the first edge and the second edge of the first band.

In some implementations, when the cable is connected to the connector port the cable provides a physical interference from wrapping the bands around the user. In some implementations, the connector port is closer to the first surface than the second surface. In some implementations, the system includes a second connector port disposed on a second side of the plurality of sides opposite of the connector port and configured to be removably connected to a second cable providing power to the power source such that when the second cable is connected to the connector port the second cable provides a physical interference from wrapping the bands around the user.

In some implementations, the second connector port is closer to the first surface than the second surface. In some implementations, the power source is configured to receive power when a cable is removably connected to the port concurrently with the second cable connected to the second port. In some implementations, the system includes a second power source disposed in the interior space, wherein the second power source is configured to be provided power when the second cable is connected to the second port.

In some implementations, the system includes a wireless magnetic sensor disposed on the second surface and configured to magnetically attach to a wireless sensing pad, wherein the wireless magnetic sensor determines whether the device is connected to the wireless magnetic pad and removed from the person before the device is charged. In some implementations, the wireless sensing pad is configured to charge the device.

Disclosed herein is a wearable device, the charging system including: a body having a first surface opposite a second surface in contact with a skin surface of a person, the first surface and second surface defining an interior space, wherein a power source is disposed in the interior space; a first band couples to a first portion of the body and a second band coupled to a second portion of the body, wherein the first band and the second band are configured to wrap around a body part of a user; and a connector port configured to be removably connected to a cable and in a proximate area to at least one of the bands, wherein the cable inhibits the first and second band from wrapping around the body part of the user.

In some implementations, the connector port is disposed on the first surface. In some implementations, the connector port is disposed on a side of the plurality of sides. In some implementations, the connector port is closer to the first surface than the second surface. In some implementations, the system includes a second connector port disposed on a second side of the plurality of sides opposite of the connector port and configured to be removably connected to a second cable providing power to the power source such that when the second cable is connected to the second connector port the second cable provides a physical interference from wrapping the bands around the user.

In some implementations, the second connector port is closer to the first surface than the second surface. In some implementations, the power source is configured to receive power when the cable is connected to the port concurrently with the second cable connected to the second port. In some implementations, the system includes a second power source disposed in the interior space, wherein the second power source is configured to be provided power when the second cable is connected to the second port.

In some implementations, the system includes a wireless magnetic sensor disposed on the second surface and configured to magnetically attach to a wireless sensing pad, wherein the wireless magnetic sensor determines whether the device is connected to the wireless sensing pad and removed from the person before the device is charged. In some implementations, the wireless sensing pad is configured to charge the device.

Disclosed herein is a device configured to be secured to a user, the device including: a body; a band configured to secure the body to a limb of the user; and a charging port located underneath a connecting portion configured to secure the band to the body, the charging port facing a direction that intersects the band.

In some implementations, the body includes a first surface opposite a second surface in contact with a skin surface of a person, the first surface and second surface defining an interior space, wherein a power source is disposed in the interior space. In some implementations, said band is connected to a first portion at a first location; and said charging port is positioned at a second location on said housing that is between said first location and a surface of the housing configured to contact skin of the user's body when the first band secures the body portion to the user. In some implementations, said first and second location are substantially vertically aligned with one another.

In some implementations, the charging port is configured to be removably connected to a cable, wherein the cable inhibits the first and second band from wrapping around the body part of the user, and wherein the cable is at least one of a power cable and a data transfer cable. In some implementations, when the cable is connected to the charging port and the body portion is positioned adjacent the user's body, the cable providers a physical interference that inhibits said band from wrapping around said limb of the user. In some implementations, the device includes a second band connected to a third portion of the housing, said second band configured to secure the body portion to the user by wrapping around the limb of the user and buckling to the first band.

In some implementations, the device includes a second charging port configured to removably connect to another cable to allow charging of said power source, said second charging port positioned at a fourth portion of the housing relative to said third portion of the housing. In some implementations, when another cable is connected to the charging port and the body portion is positioned adjacent the user's body, the cable provides a physical interference that inhibits said second band from wrapping around said limb of the user.

In some implementations, the power source is configured to be charged when the cable is connected to the charging port concurrently with another cable connected to the second charging port. In some implementations, the device includes a second power source disposed in the interior, wherein the second power source is configured to be charged when the another cable is connected to the second charging port.

In some implementations, the device includes a wireless magnetic sensor disposed on the housing and configured to magnetically attach to a wireless pad, wherein the wireless magnetic sensor determines whether the body portion is located on the wireless magnetic pad before the power source is charged. In some implementations, the wireless magnetic pad is configured to charge the power source.

Disclosed herein is a wearable device for determining one or more physiological parameters indicative of multivariate stroke, the wearable device including: a body portion including: a housing including a first surface, a second surface opposite said first surface, a plurality of side surfaces extending between the first and second surfaces, an interior, and a battery positioned within the interior; a first band connected to a first side of the plurality of sides of the housing, said first band configured to secure the body portion to a user by wrapping around at least a portion of the user's body, wherein said second surface of said housing is configured to contact skin of the user's body when the first band secures the body portion to the user; and a connector port configured to removably connect to a cable to allow charging of said battery, said connector port positioned at a location on said first side of the housing such that, when the cable is connected to the connector port and said second surface of said housing is positioned to contact said skin, the cable provides a physical interference that inhibits said first band from wrapping around said at least a portion of the user's body.

In some implementations, said first band is connected to said first side at a first location; and said connector port is positioned at a second location on said first side that is between said first location and said second surface of the housing. In some implementations, said first and second location are substantially vertically aligned with one another. In some implementations, the cable is at least one of a power cable and a data transfer cable.

In some implementations, the wearable includes a second band connected to a second side of the plurality of sides of the housing, said second band configured to secure the body portion to the user by wrapping around at least a portion of the user's body and buckling to the first band. In some implementations, the wearable device includes a second connector port configured to removably connect to a cable to allow charging of said battery, said second connector port positioned at a location on said second side of the housing such that, when the cable is connected to the connector port and said second surface of said housing is positioned to contact said skin, the cable provides a physical interference that inhibits said second band from wrapping around said at least a portion of the user's body.

In some implementations, the battery is configured to be charged when the cable is connected to the connector port concurrently with the second cable connected to the second connector port. In some implementations, the wearable device includes a second battery disposed in the interior, wherein the second battery is configured to be charged when the second cable is connected to the second connector port.

In some implementations, the techniques described herein relate to a wearable device, further including a wireless magnetic sensor disposed on the second surface and configured to magnetically attach to a wireless magnetic pad, wherein the wireless magnetic sensor determines whether the body portion is located on the wireless magnetic pad before the device is charged. In some implementations, the wireless magnetic pad is configured to charge the battery.

Disclosed herein is a charging system for a wearable device, the charging system including: a body having a first surface opposite a second surface in contact with a skin surface of a person, the first surface and second surface defining an interior space, wherein a power source is disposed in the interior space; a plurality of sides disposed between the first surface and the second surface; a first band coupled to a first side of the plurality of sides, the first band including a first edge and a second edge opposite of the first edge defining a width of the first band; a second band coupled to a second side of the plurality of sides opposite of the first band, the second band including a first edge and a second edge opposite of the first edge defining a width of the second band wherein, the first band and the second band are configured to wrap around a portion of a user; a connector port disposed on a side surface of the plurality of sides configured to be removably connected to a cable providing power to the power source such that when the cable is connected to the connector port the cable provides a physical interference from wrapping the bands around the user.

In some implementations, when the cable is connected to the connector port the cable provides a physical interference from wrapping the bands around the user. In some implementations, the connector port is closer to the first surface than the second surface.

In some implementations, the system includes a second connector port disposed on a second side of the plurality of sides opposite of the connector port and configured to be removably connected to a second cable providing power to the power source such that when the second cable is connected to the connector port the second cable provides a physical interference from wrapping the bands around the user. In some implementations, the second connector port is closer to the first surface than the second surface.

In some implementations, the power source is configured to receive power when a cable is removably connected to the port concurrently with the second cable connected to the second port. In some implementations, the system includes a second power source disposed in the interior space, wherein the second power source is configured to be provided power when the second cable is connected to the second port.

In some implementations, the system includes a wireless magnetic sensor disposed on the second surface and configured to magnetically attach to a wireless sensing pad, wherein the wireless magnetic sensor determines whether the device is connected to the wireless magnetic pad and removed from the person before the device is charged. In some implementations, the wireless sensing pad is configured to charge the device.

Disclosed herein is a wearable device configured to be secured to a user's body, the device including: a body portion including a housing, an interior within the housing, and a battery positioned within the interior; a band connected to a first portion of the housing, said band configured to secure the body portion to the user by wrapping around at least a portion of the user's body; and a connector port configured to removably connect to a cable to allow charging of said battery, said connector port positioned at a second portion of the housing relative to said first portion of the housing such that, when the cable is connected to the connector port and the body portion is positioned adjacent the user's body, the cable provides a physical interference that inhibits said band from wrapping around said at least the portion of the user's body.

In some implementations, said band is connected to said first portion at a first location; and said connector port is positioned at a second location on said housing that is between said first location and a surface of the housing configured to contact skin of the user's body when the first band secures the body portion to the user. In some implementations, said first and second location are substantially vertically aligned with one another.

In some implementations, said band is connected to said first portion at a first location; and said connector port is positioned at a surface of the housing configured to contact skin of the user's body when the first band secures the body portion to the user. In some implementations, the cable is at least one of a power cable and a data transfer cable.

In some implementations, the techniques described herein relate to a device to 139, further including a second band connected to a third portion of the housing, said second band configured to secure the body portion to the user by wrapping around at least a portion of the user's body and buckling to the first band.

In some implementations, the techniques described herein relate to a device, further including a second connector port configured to removably connect to a cable to allow charging of said battery, said second connector port positioned at a fourth portion of the housing relative to said third portion of the housing such that, when the cable is connected to the connector port and the body portion is positioned adjacent the user's body, the cable provides a physical interference that inhibits said second band from wrapping around said at least the portion of the user's body.

In some implementations, the battery is configured to be charged when the cable is connected to the port concurrently with the second cable connected to the second connector port. In some implementations, the device includes a second battery disposed in the interior, wherein the second battery is configured to be charged when the second cable is connected to the second connector port. In some implementations, the device includes a wireless magnetic sensor disposed on the housing and configured to magnetically attach to a wireless pad, wherein the wireless magnetic sensor determines whether the body portion is located on the wireless magnetic pad before the battery is charged. In some implementations, the wireless magnetic pad is configured to charge the power source.

Disclosed herein is a system for monitoring a user during a stimulus cycle, the system including: a wearable device including: a heating source; one or more sensors, wherein the one or more sensors are configured to sense a physiological parameter during the stimulus cycle; and one or more electrodes configured to turn on and off based at least on which of the one or more sensors is receiving data; wherein the stimulus cycle includes at least one of a baseline duration, a ramp-up duration, a dwell duration, and a post duration.

In some implementations, the baseline duration includes a measuring of different signals. In some implementations, the different signals include at least one of electrodermal activity (EDA), electromyograph (EMG), and impedance cardiography (BioZ).

In some implementations, the different signals correspond to the one or more electrodes, wherein the one or more electrodes are switched on or off depending on the different signal during the stimulus cycle. In some implementations, the switching on or off of the one or more electrodes occur contemporaneously. In some implementations, the techniques described herein relate to a system, wherein the one or more sensors are configured to multiplex a signal received from each of the one or more sensors. In some implementations, the system includes any of the features mentioned herein.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various implementations, with reference made to the accompanying drawings.

FIG. 1A illustrates an example of a wearable device for monitoring physiological parameters of a user.

FIG. 1B illustrates a block diagram of an example physiological monitoring system for obtaining and processing biological data.

FIG. 1C illustrates a block diagram of an example computing environment for processing biological data.

FIG. 2 illustrates a flow diagram of an example process for generating biological data characterizations and/or estimates.

FIG. 3 illustrates a first and second wearable device for measuring biological data and response asymmetry across a right and left limb, respectively.

FIG. 4A illustrates a perspective view of one implementation of a wearable device for obtaining biological data.

FIG. 4B illustrates a perspective view of the wearable device of FIG. 4A.

FIG. 4C illustrates a partial view of the wearable device of FIG. 4A.

FIG. 4D illustrates a rotated view of the wearable device of FIG. 4C.

FIG. 4E illustrates a perspective view and partial exploded view of the wearable device of FIG. 4C.

FIG. 4F illustrates a front view of the wearable device of FIG. 4C.

FIG. 5A illustrates a schematic diagram of a temperature measuring system for a wearable device.

FIG. 5B illustrates an exemplary implementation of the temperature measuring system of FIG. 5A.

FIG. 6 illustrates an exemplary implementation of a hardware protection circuit to be used in FIG. 5A.

FIG. 7 illustrates a flow diagram of an example process for estimating vasodilation activity based on electrodermal activity (EDA), electromyograph (EMG), and impedance cardiography (BioZ) signals.

FIGS. 8 and 9 illustrate example timelines of the example process of FIG. 7.

FIGS. 10 and 11 illustrate another example of a wearable device for monitoring physiological parameters of a user.

FIG. 12 illustrates the wearable device of FIGS. 10-11 connected to a cable.

The illustrated implementations are merely examples and are not intended to limit the disclosure. The schematics are drawn to illustrate features and concepts and are not necessarily drawn to scale.

### I. DETAILED DESCRIPTION

Disclosed herein are systems and methods for preventing burns of a user when obtaining, monitoring, and/or characterizing a vasodilation response of said user. For example, the devices may include wearable devices configured to stimulate a skin surface site to obtain the vasodilation response of a user wearing such devices. PCT Application PCT/US2022/071701, incorporated herein in its entirety, shows example wearable systems and methods for measuring vasodilatory response.

Monitoring for stroke symptoms or physiological symptoms using a stimulus source contacting a skin surface of a user presents some risks should the stimulus source suffer a failure. Such failures during stimulus cycles may lead to bodily injury or worse as the stimulus source may approach harmful temperatures exceeding pain thresholds. Typical safety systems may allow the system to operate if the circuits are closed. Safety circuits can be comprised of components wired in series to maximize effectiveness and minimize complexity and cost. Thus, if a component is triggered, the whole safety circuit will remain open, and the device will not work.

The warming and cooling of a detection cycle present inherent dangers when in close proximity to exposed skin surfaces. As the stimulus source increases in temperature, any failures in the control logic of the stimulus source provides an opportunity for injuries or pain. Several governing standards provide temperature limits for touchable circuits. For example, ASTM C1055-20 indicates that the pain threshold occurs at approximately 44 degrees Celsius, with IEC60950-1/UL62368-1 indicating that the permitted temperature as a function of expose time for parts exposed to skin for prolonged use is not permitted to exceed 48 degrees Celsius. Additionally or alternatively, as determined in some cases, a cut off of approximately 42 degrees Celsius may be used. At approximately 42 degrees Celsius, the vasodilation response of the user in contact with a heater may plateau. Any further increases in temperatures do not elicit a change in the vasodilator response.

In operation, the devices and systems described herein can disconnect a heating stimulus from a power source in response to one or more fault conditions when the device is obtaining physiological data from a skin surface site of a person to estimate vasomotor responses (e.g., vasodilation activities, responses, levels, signals, parameters, time-based parameters, etc. and/or vasoconstrictive activities, responses, levels, signals, parameters, time-based parameters, etc.) of the person.

The systems and methods described herein may solve a technical problem of preventing tissue injuries to exposed skin surfaces of a user during stimulus cycles for generating ambulatory and/or home-based (i.e., not in a medical facility) estimates for vasodilation activity using a wearable device to enable capture of one or more physiological signals. The systems and methods described herein may provide an example technical solution to the technical problem by providing a temperature measuring system in a wearable device for obtaining physiological signal data and generating a number of increasingly accurate vasodilation activity estimates for a particular skin surface site.

Additionally, the systems and methods described herein may solve a technical problem of generating reproducible and/or substantially continuous estimates for vasodilation activity using a wearable device to enable capture of one or more physiological signals. The systems and methods described herein may provide an example technical solution to the technical problem by providing a wearable device for obtaining physiological signal data and generating a number of increasingly accurate vasodilation activity estimates for a particular skin surface site during a stimulus cycle.

The estimates described herein include vasodilation activity based on physiological signal(s) that may be continuously or intermittently obtained by the wearable device 102. Obtaining the physiological signal(s) may automatically a trigger device 102 to generate estimates for vasodilation levels (e.g., vasodilation activity) for one or more blood vessels associated with the skin surface site that is in contact with (or near to) a portion of the wearable device 102. The technical solution improves vasodilation capture and estimation over conventional solutions (e.g., ultrasound technology) because the systems and methods described herein may provide a way to assess vasodilation without the expense, inconvenience, and expertise of ultrasound technology.

### Wearable Physiological Monitoring Device

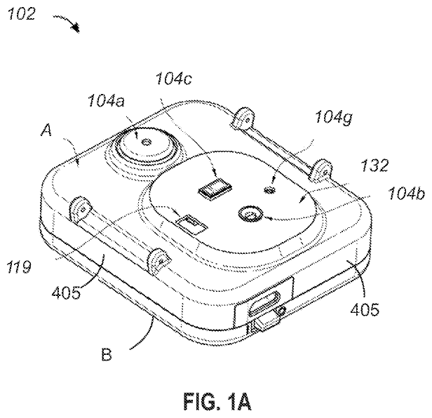

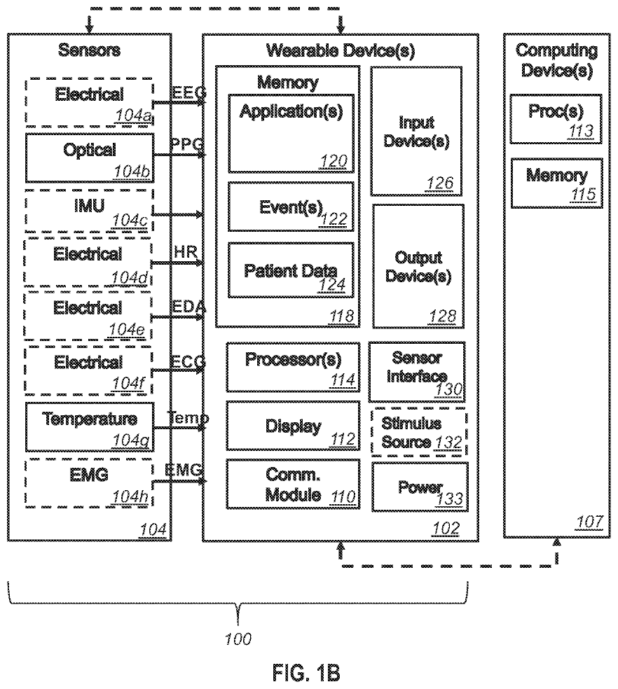

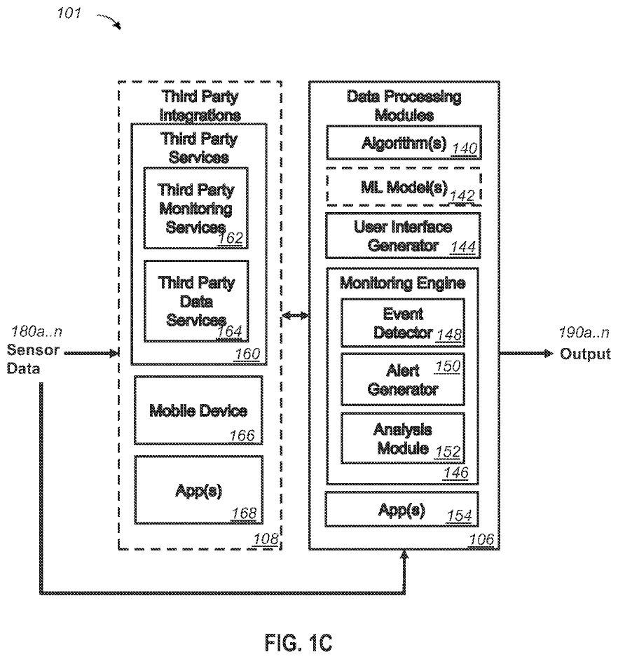

FIG. 1A illustrates an example of a wearable device 102 for monitoring physiological parameters of a user (e.g., a person, a patient). The wearable device 102 can include one or more sensors for detecting physiological parameters of the user. For example, the wearable device 102 can include an electrical sensor 104a for obtaining temperature responses and/or EDA responses from a skin surface site of the user. The wearable device 102 can also include an optical sensor 104b for obtaining physiological measurements from a skin surface site of the user. Other sensors and/or elements may be incorporated into or near to the device 102, as will be described in detail below.

The wearable device 102 may be configured to measure any number of physiological parameters of a user wearing the device 102. The measurements may be retrieved using one or more sensors integrated into or associated with device 102. For example, the device 102 may include one or more sensors for measuring core and/or skin surface temperatures; volumetric impedance spectroscopy; hyperhidrosis; heart rate or heart rate variability; and/or motion (e.g., by including an accelerometer and/or gyroscope therein) to measure, for example, limb asymmetry or changes in gait. Alternatively, the device 102 may be communicatively coupled (e.g., via antenna, coils, etc.) to an external sensor that is not housed in or integrated into device 102.

Some or all components of the wearable device 102 may be integrated into a patch, a band, a watch, an adhesive strip, a bracelet, an anklet, a sock, a shoe insole, a shoe, clothing, or any other wearable accessory. For example, some or all components of the wearable device 102 may be incorporated into a ring or band or a pair of rings or bands to be worn one on each ankle or each foot. In some implementations, the ring or band may incorporate a stretchable or expandable element or stretch sensor to allow the ring or band to expand or stretch when a body portion near the ring or band swells. This element may include, but is not limited to, elastomer film polymers of various degree of bonding to allow for different pliable elements or measuring the reflectivity of polarized light. This element may include a plastic segment of the ring or band that can be loosened/tightened, or by building a slidable element that can be pulled apart. Non-limiting examples of a stretch sensor include, but are not limited to, a strain gauge or an electrical component which can change inductance, resistance, or capacitance when stretched.

Referring again to FIG. 1A, the wearable device 102 includes a body portion with a first surface A opposite a second surface B. The first surface A may be placed in contact with a skin surface of a user to obtain measurements and provide a stimulus via a stimulus source 132, for example heat via a heat source. The first surface A and second surface B may be coupled via one or more or a plurality of sidewalls of device 102. For example, one or more sidewalls 405 may extend from a perimeter of the first surface A and couple to a perimeter of the second surface B. The first surface A and/or second surface B may include one or more sensors 104 (e.g., sensor or electrode 104a, sensors 104b, 104c, 104d, 104e, 104f, 104g, and/or 104h, as shown in FIGS. 1A-1B) positioned thereon. For example, one or more sensors 104 on the first surface A may measure one or more properties, features, or characteristics of the skin surface of the user, and one or more sensors on the second surface B may measure an environment of the user wearing or using the wearable device 102. In some implementations, the wearable device 102 may include an opening, such as opening 119, to allow the heat source 132 to communicate with and/or access other components inside the body of device 102, for example processor(s) 114 and/or power source 133.

The wearable device 102 may be worn on an exterior or skin surface of a user (e.g., a patient). For example, the wearable device 102 may be configured to be positioned on a body portion, and the one or more electrodes or sensors may be configured to be positioned on a region of the body portion or a region near to the body portion. In some implementations, the wearable device 102 may be worn on an exterior or skin surface of a user (e.g., a patient) prior to, during, and/or after an anomalous biologic event, including up to days before the event, during the event, and/or after the event to provide continuous variable monitoring of various physiological parameters. For example, the wearable device 102 may function to monitor or characterize a vasodilation response in response to application of a stimulus (heating or cooling) or as a result of fluctuations in vasodilation.

In some implementations, the wearable device 102 may include a strip or form a strip that measures muscle contractions through surface electromyography (sEMG). The measurement of EMG may be compared to a baseline value to detect a change or asymmetry of the EMG. In some implementations, EMG measures a user's intent to move a muscle and a corresponding actual user movement of the same muscle, compared to a typical movement profile of the muscle or a movement response on an opposite limb or extremity.

In some implementations of device 102, one or more electrodes 104a may be integrated into a body of the wearable device 102 or in a separate component of the wearable device 102. Further, in some implementations of the device 102, a stimulus source 132 is not utilized when monitoring and/or characterizing a vasodilation response. In a non-limiting example of a wearable device without a stimulus source 132, fluid shift within the vasculature (e.g., when someone moves from laying to sitting to standing) may be sufficient to detect vasodilation as a result of the fluid shift. For example, the wearable device 102 may detect limb position or perform pulse wave analysis, to evaluate aspects of vasodilation. In one exemplary, non-limiting embodiment, when the arm is up in the air, the blood drains out of the hand resulting in the photoplethysmogram alternating current (AC) amplitude dropping. With the arm down by the side, the AC amplitude may increase because of blood pooling in the hand. The same effects occur in the feet when moving between standing, sitting, and laying down due to gravity induced blood pooling. A communicatively coupled accelerometer or an accelerometer embedded in the wearable device 102 may be used to detect limb position so that limb position may be tracked and a blood volume signal recorded based on the detected position.

In some implementations, the stimulus source 132 is not included in the wearable device 102. Instead, ambient environment temperature fluctuations that naturally alter vasodilation at one or more skin surface sites may be used to detect vasodilation. An ambient temperature fluctuation may include hot or cold water, under the covers, natural sunlight, warm or cold outdoors, and the like. Alternatively, the stimulus source 132 may be an external stimulus source, for example a laser. For example, when the wearable device 102 does not include a stimulus source 132, a processor of the wearable device 102 may receive a first skin temperature at time A and may receive a second skin temperature at time B to compare the underlying vasomotor tone of the blood vessels. Internal studies show that core body heating due to exercise increases the vasodilation output without any external heating stimulus. In such instances, blood volume measurements may be substantially continuous rather than a stimulus-based response.

In some implementations, the wearable device 102 may generate a stimulus for delivery to a monitoring site and measure a response in physiological parameters based on the stimulus. The stimulus may be applied to one location or a plurality of locations. In one implementation, the stimulus is applied bilaterally (e.g., to detect asymmetrical responses) on the body of the user to determine whether the response or the difference in response between the two sides indicates an atypical event, a stroke event, or a deviation from baseline. For example, the stimulus may be applied in a stimulus cycle such that the baseline, during stimulation, and post stimulation responses are measured, or change in (e.g., slope, decay, etc.) responses between different measurement periods are determined. For example, a thermal (i.e., hot or cold) stimulus may be applied to a section of skin on a body of a user (shown in top panel) and the body's response to the thermal stimulus may be monitored over time (shown in bottom panel) to determine whether homeostasis is reached and/or a difference in response or return rate exists between the two sides of the body (in other words, determine whether an asymmetrical response exists). Responses can be indicative of changes in or perturbations in the parasympathetic nervous system, the sympathetic nervous system, the central vascular system, or the peripheral vascular system.

Further examples include stimulating the muscular or nervous system using electrical signals and monitoring the response over time and/or between sides using electromyogram (EMG), bioimpedance, or electroneurogram (ENG), respectively. These “stimulators/transmitters” and “receivers/detectors” could be in the same region or could be separated to measure across regions of the body.

Applying heat stress to a portion of the skin may enable detection of a vasodilation response. Accordingly, systems and methods described below may enable detection of impaired vasodilation in a form factor that improves continuous anomalous cardiac event monitoring. In some implementations, the wearable device 102 may function to heat a skin surface and measure a vasodilation response of the skin surface. The device 102 may further function to measure one or more additional parameters, biologic signals, etc. as will be described in greater detail elsewhere herein. In some implementations, the device 102 may use a measured bioimpedance (BioZ) to validate or invalidate a vasodilation measurement from another sensor on device 102.

In some implementations, the device 102 may include and/or communicate with a device positionable in a room, office, home, vehicle, or other location; or in or on a bed or other furniture (e.g., bedside monitors; monitors within mattresses, bedding, etc.). For example, a smart speaker (e.g., to prompt a user to respond to a question to analyze speech quality), microphone, camera, and/or mirror may be positionable in a location to detect changes in a user's speech, activities, movement, gait, facial appearance, heart rate, and/or heart rate variability or changes from baseline. The device may include one or more data processing modules to differentiate changes in the measured parameters as compared to that from healthy learned patient data or individualized baseline data. The wearable device 102 may be worn on an exterior or skin surface of a user (e.g., a patient) prior to, during, and/or after an anomalous biologic event, including up to days before the event, during the event, and/or after the event to provide continuous variable monitoring of various physiological parameters. For example, the wearable device 102 may function to monitor or characterize a vasodilation response in response to application of a stimulus (heating or cooling) or as a result of fluctuations in vasodilation (without a stimulus source).

### Physiological Monitoring System

FIG. 1B illustrates a block diagram of a physiological monitoring system 100. The system 100 can include a device 102 as described above and one more sensors 104. The system 100 may measure, characterize, or detect physical responses (e.g., stroke events, stress response, heat stroke, seizure, menopause, diabetes, etc.) for a user wearing a wearable device that includes (or is in communication with) monitoring system 100. In some implementations, the sensors 104 can be integrated with the device 102. In additional implementations, some or all of the sensors 104 may be physically separate from the device 102. The sensors 104 can be communicatively coupled to the wearable device 102 including wired and/or wireless connections. The system 100 may also connect with a computing device 107 as described below. Alternatively, or additionally, the system 100 may integrate with, be communicatively coupled to third-party devices and/or services and/or processing system 101 (FIG. 1C). In some embodiments, the processing system 101 is incorporated into system 100.

In some implementations, the wearable device 102 can include a communication module 110, a display 112, processors 114, and memory 118. The display 112 may not be included in all implementations. The communication module 110 may include one or more antennas or coils for wireless connections. The communication module 110 may also include components for wired connection, such as USB data transfer.

The processors 114 may include one or more hardware processors, including microcontrollers, digital signal processors, application specific integrated circuit (ASIC), a field programmable gate array (FPGA) or other programmable logic device, discrete gate or transistor logic, discrete hardware components, or any combination thereof designed to perform the functions described herein and/or capable of executing instructions, such as instructions stored by the memory 118. The processors 114 may also be able to execute instructions for performing communications amongst wearable device 102, sensors 104, data processing modules 106, and/or third-party integration 108.

The memory 118 can include one or more non-transitory computer-readable storage media. The memory 118 may store instructions and data that are usable in combination with processors 114 to execute algorithms 140, optional machine learning models 142, monitoring engine 146 tasks, and apps 154. The memory 118 may also function to store or have access to the data processing modules 120, events 122, and patient data 124.

The system 100 may further include or be communicatively coupled to input devices 126, output devices 128, sensor interface 130, stimulus source 132, and/or power (e.g., battery) 133. The input devices 126 may interact with one or more processors 114, memory 118, and/or sensors 104. The input devices 126 may include buttons, touchscreens, switches, toggles, and/or other hardware components located on wearable device 102. In some implementations, the input device 126, or at least some functionality of input device 126, may be external to or not integrated into the device 102, such that one or more controllers, mobile device (e.g., mobile device 166), apps (e.g., apps 168), etc., may communicate (e.g., antenna, coils, etc.) with device 102 using a wireless communications protocol. In some implementations, the input device 126 may include, for example, a touch input device that can receive tactile user input, a microphone that can receive audible input, and the like.

The output devices 128 may interact with one or more processors 114, memory 118, and/or sensors 104. The output devices 128 may include, for example, a display (e.g., display 112) for visual output, a speaker for audio output, and/or haptic feedback.

The sensor interface 130 may store instructions to carry out operations pertaining to received sensor signals from one or more of the sensors 104. For example, the instructions may enable interaction with one or more processors 114, memory 118, and/or sensors 104 to communicate sensor data from one or more of the sensors 104 to the wearable device 102. The sensor data may be obtained from the sensors 104 taking recordings and measurements. The sensors 104 may include any or all the electrical sensors 104a, 104d, 104e, 104h, optical sensor 104b, temperature sensor 104g, and/or mechanical sensors (e.g., strain gauge, sensor 104c, etc.), as described in detail throughout this disclosure.

The stimulus source 132, in one embodiment, may be a heating element, a thin film resistance flexible heater, a polyimide or PDMS heater, an optical heater (e.g., a laser), chemical heater, and the like. In some implementations, the stimulus source 132 may be a cooling element, a thermoelectric cooler, a miniature compressor, or the like. The stimulus source 132 may be configured to be placed in communication with the skin surface. The stimulus source 132 may be configured to heat or cool the skin surface to a target offset temperature or a pre-determined temperature. In some implementations, the stimulus source 132 may not be integrated into the wearable device 102 and may instead be an environmental heat source, for example a warm room, warm enclosure, and/or other warm environment.

Skin temperature measurement devices that may be installed within and/or communicatively coupled to device 102 may include, but are not limited to, infrared sensors, thermometers, thermistors, or thermal flux transducers.

Hyperhidrosis measurement devices may include, but are not limited to, detection of analytes including ions, metabolites, acids, steroids (e.g., hormones), and proteins through potentiometry, chronoamperometry, cyclic voltammetry, square wave stripping voltammetry, or detection of changes in conductivity.

The power 133 may be a power source for the wearable device and/or sensors 104. The power 133 may include or connect to a battery or a port for connecting the device 102 to a power supply, a wall power adapter, or the like.

The sensors 104 may function with one or more processors 114, memory 118 to obtain and communicate sensor signal data to display 112, data processing modules 106, and/or third-party integrations 108. The sensors may include one or more of an electroencephalogram (EEG) sensor 104a, a blood volume sensor 104b (e.g., a photoplethysmographic (PPG) sensor), an inertial measurement unit (IMU) sensor 104c, a heart rate (HR) sensor 104d, an electrodermal activity (EDA) sensor 104e, an electrocardiogram (ECG) sensor 104f, a temperature sensor 104g (e.g., a skin temperature sensor), and an electromyography (EMG) sensor 104h and/or mechanical sensors (not shown).

Sensor measurement devices that may be installed within and/or communicatively coupled to device 102 may include, but are not limited to, a photoplethysmographic (PPG) device, a skin conductance sensor measuring skin conductance/galvanic skin response (GSR) or electrodermal activity (EDA), or a skin temperature measurement device (e.g., contact devices and non-contact devices, like IR imaging camera).

The EEG device or component of a device 104a may include one or more electrodes configured to detect normal and abnormal changes in the electrical activity of the brain.

The blood volume sensor 104b may include an optical sensor to detect blood volume changes in tissue. The blood volume sensor 104b may measure volumetric impedance spectroscopy, heart rate, respiration rate, or heart rate variability through monitoring a rate of blood flow.

The IMU sensor 104c may monitor motion (e.g., by including an accelerometer and/or gyroscope therein) to measure, for example, limb asymmetry or changes in gait or limb movement.

The heart rate sensor 104d may include one or more electrical sensors (e.g., electrocardiogramal sensors (e.g., PPG), or mechanical sensors (e.g., accelerometer, strain gauge, pressure sensors) to measure and monitor a heart rate.

The EDA sensor 104e may measure hyperhidrosis. Hyperhidrosis measurement devices may include, but are not limited to, evaluating sweat rate, detection of analytes including ions, metabolites, acids, hormones, and small proteins through potentiometry, chronoamperometry, cyclic voltammetry, or detection of changes in conductivity.

The ECG sensor 104f may include a plurality of electrodes for recording electrical signals of the heart, to determine heart rate, determine cardiac rhythm, cardiac autonomic control, and other cardiovascular and cardiorespiratory metrics. In instances of a stroke event, changes in these signals may indicate a potential stroke state.

The temperature sensor 104g may measure temperature. Temperature sensors may include, but are not limited to, infrared sensors, infrared imaging cameras, thermometers, thermistors, or thermal flux transducers. Temperature sensors are used to measure the core body temperature and surface temperatures at various sites on the body. In a stroke event, there may be variations in temperature related to disruptions in temperature regulation, both at the core as well as at individual surface sites, that may indicate a stroke event.

The EMG sensor 104h may include two or more or a plurality of electrodes for recording electrical signals of muscles. For example, an EMG sensor 104h may be used to measure an intent to move signal and a resulting movement signal of a muscle. In instances of a stroke event, the intent to move signal may be recorded but the movement signal may be absent, indicating a potential stroke state.

Additional metrics that may be obtained by device 102 with one or more additional sensors 104, processors 113 and/or processors 114, and/or electrodes may include, movements, reflexes, stimulus response output, breathing patterns, metrics responsive to audio input, etc.

In some implementations, the wearable device 102 may include or be communicatively coupled to a computing device 107. The computing device 107 may be a mobile device, a laptop device, a second wearable device, or the like. The computing device 107 may be a computer-executable component that includes processors 113 and memory 115 that may be used to execute the methods and described herein. In some implementations, the computing device 107 represents a general or application-specific hardware processor, but any suitable dedicated hardware or hardware/firmware combination can alternatively or additionally execute the instructions.

In some implementations, the system 100 or environment 101 (operating on device 102) may further include an application (e.g., application 120) downloaded and/or stored on a hardware component of device 102 or on a hardware component of computing device 107. The application may be configured to process sensor data, electrode data, camera data, speech data, and/or display data sensed or captured in real time, for example in a graphical representation, of the data.

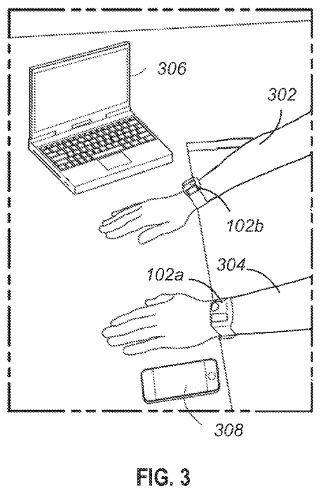

While the system 100 is depicted with a single wearable device 102, any number of wearable devices 102 may be included or communicatively coupled to system 100 (e.g., a first wearable device 102a and/or a second wearable device 102b of FIG. 3). In some implementations, the system 100 may operate on wearable device 102 to perform monitoring and event detection. In some implementations, the system 100 may operate external to wearable device 102 but may communicate with device 102 to obtain measurements and provide analysis of such measurements.

Data obtained with wearable device 102 may be transmitted to and/or from the system 100 (and/or environment 101 and/or computing device 107) to a central hub, mobile computing device (e.g., computing device 107), server, or other storage and/or computing device. Sharing of such data is performed according to user permissions. For example, a user of device 102 may configure data sharing permissions for device 102 to ensure that the data being shared is shared according to the configured user permissions.

Data transmissions described herein may include wireless communication (e.g., a nearfield communications (NFC) protocol, a low energy Bluetooth® protocol, other radiofrequency (RF) communication protocol, etc.) between sensor locations on the body and/or a central hub. In some implementations, data transmission may include wire communication between sensor locations on the body and/or a central hub. In some implementations, the central hub may be a monitor in a medical facility, home monitor, patients' mobile computing device, or other wireless device. Alternatively, one or more of the sensors on the body may act as the central hub. The hub device may wirelessly send signals to activate a medical care pathway and/or notify one or more individuals (e.g., family, friends, physician, EMS, etc.). In some implementations, data transmission, following multivariate analysis, to the central hub may alert the patient, the next of kin, and/or a third party to identify possible false positives or negatives.

### Processing System(s)

FIG. 1C illustrates a block diagram of an example computing environment 101 for processing biological data. Some or all aspects of the computing environment 101 may be implemented by the systems and devices described above. As shown, the computing environment 101 includes data processing modules 106 and optional (shown by dashed lines) third-party integrations 108. The data processing modules 106 can include multiple engines for performing the processes and functions described herein. The engines can include programmed instructions for performing processes as discussed herein for detection of input conditions and control of output conditions. The engines can be executed by the one or more hardware processors of the device 102 alone or in combination with other hardware devices, such as the computing device 107. The programming instructions can be stored in a memory as discussed above. The programming instructions can be implemented in C, C++, JAVA, or any other suitable programming languages. In some embodiments, some or all of the portions of the data processing modules 106 including the engines can be implemented in application specific circuitry such as ASICs and FPGAs. Some aspects of the functionality of the controller associated with data processing modules 106 can be executed remotely on a server (not shown) over a network. While shown as separate engines, the functionality of the engines as discussed below is not necessarily required to be separated. Accordingly, the data processing modules 106 can be implemented with the hardware components described above with respect to FIGS. 1A-1B.

In operation, the data processing modules 106 and/or the third-party integrations 108 may receive one or more inputs (input sensor data 180a through 180n (e.g., 180a . . . 180n)), and may process the inputs and generate output data 190a through 190n (e.g., 190a . . . 190n). For example, the data processing modules 106 and/or the third-party integrations 108 may receive sensor data from electrical sensor 104a and the optical sensor 104b may process the received data via algorithms 140, ML models 142, and/or monitoring engine 146 to generate a vasodilation estimation for display on an app 154. To carry out the processing, one or more data processing modules 106 may interact with one or more processors 114, memory 118, and/or sensors 104 to determine a vasodilation response and/or related physiological responses. Monitoring and/or characterizing vasodilation responses may result in indications of biological changes, such as menopause, or anomalous biologic events, such as stroke, diabetes, peripheral blood circulation disorders, etc.

As shown in FIG. 1C, the data processing modules 106 include algorithms 140, optional ML models 142, user interface generator 144, monitoring engine 146, and apps 154. The monitoring engine 146 can further include an event detector 148, an alert generator 150, and an analysis module 152. In some implementations, the data processing modules 106 may be stored and executed as data processing modules 120 (FIG. 1B) on wearable device 102.

The algorithms 140 may include computer executable code adapted to carry out any of the methods described herein. In some implementations, the algorithms 140 utilize third party integrations 108 and/or optional ML models 142 to generate output. In operation, the algorithms 140 may operate on particular sensor data 180a . . . 180n (e.g., obtained from sensors 104 on device 102). The operations may prepare the data (e.g., preprocess) and/or otherwise analyze the data to generate estimates (e.g., PPG, respiration rate, heart rate, event occurrence, etc.). The data processing modules 106 can control operations of the device 102. For example, the data processing modules 106 can output a first activation signal to an electronic stimulus source.

The optional ML models 142 may use machine learning techniques to estimate vasodilation activity for blood vessels associated with a particular skin surface site. For example, the ML models 142 may perform analysis, pattern classification, and/or recognition algorithms on PPG signals to assess changing optical properties of underlying tissues triggered by a stimulus provided by a wearable device. Portions of the analyzed signals may be used to generate vasodilation activity estimates. In some implementations, the ML models 142 may operate on signals obtained from sensors 104. The operations may include signal processing that may employ signal processing tools, for example filtering, extracting, digitizing, data de-convolution, machine learning, and/or other methods known in the art. Specifically, the signal processing may use higher order statistics to ascertain hidden patterns in data. Use of higher order statistics, known as cumulants, and their Fourier spectra, often termed poly spectra, not only reveal the amplitude information in the higher order (such as those carried by power spectra or auto correlation) but may also include phase information. Phase information can reveal salient features of the data, otherwise unattainable from simple harmonic analysis.

The user interface generator 144 may interact with one or more processors 113, 114, memory 115, 118, and/or sensors 104. The user interface generator 144 may generate user interfaces for display to a user of the wearable device 102 or other user associated with the wearable device 102. The user interfaces may include patient data, sensor measurements and data, instructions, diagnosis data, or the like. The user interface may be presented on the display 112 of the wearable device and/or on a display of a companion device such as a mobile phone, laptop, tablet, or computer configured to receive information and user interfaces from the wearable device 102. The user interface may be configured to display a vasodilation response of the user over time, relative to a population, across multiple skin sites of a user, etc. The user interface may be further configured to display additional physiological responses of the user over time, relative to a population, across multiple skin sites, etc., for example an electrodermal activity response, muscle activity, hydration state, etc. The vasodilation response and physiological response(s) may be overlaid or displayed independently depending on the intended analysis.

The monitoring engine 146 may determine when events or data changes occur at one or more of the sensors 104 (or associated electrodes). The monitoring engine 146 includes an event detector 148, an alert generator 150, and an analysis module 152. The event detector 148 may monitor and detect biological signals to monitor or characterize the signals or determine whether an anomalous biologic event has occurred. The analysis module 152 may function in combination with the event detector 148 to determine whether an event (or change in biological data) is to trigger an alert. The alert generator 150 may receive the determination of whether to trigger an alert and in response, may trigger the alert. For example, the temperature sensor 104g may function with the monitoring engine 146 to monitor and obtain temperature at a particular skin surface site associated with the wearable device 102. The monitoring engine 146 may trigger the alert generator 150 to generate an alert in response to determining that a monitored temperature has reached or exceeded a predefined threshold temperature.

The alert generator 150 may alert the user wearing the wearable device 102 and/or alert a third party of an event, available data, and/or a change in data. In some implementations, the alert may be an audible sound or a visual indicator or message to the user via the device 102 or via a mobile device, computing device associated with the device 102. In some implementations, the alert may be a message sent to emergency services or physicians. Alerting emergency services or physicians, data including medical history may be transmitted directly to emergency services or physician computing systems, either directly from the wearable device 102 or from a remote memory, initiated by a signal from the wearable device 102. In addition to alerts, the wearable device 102 can also instruct a user to undertake or automatically activate certain treatments.

The apps 154 may represent one or more software applications that may be installed on (or accessed from) the wearable device 102 (and/or an associated mobile phone or computing device 107). The apps 154 may be used to present user interfaces generated by user interface generator 144.

Optionally (shown by dashed lines), the environment 101 may further include a third-party device or integration 108, for example a device including Amazon® Alexa® or an Amazon® Echo® device, as described in further detail elsewhere herein. For example, there may be bidirectional communication (e.g., via a wired connection or wireless communication) between the hardware component and the data processing modules 106, the data processing modules 106 and the third-party device or integration 108, and/or the third-party device or integration 108 and the hardware component.

Third party integrations 108 may optionally function with the wearable device 102, sensors 104, and/or data processing modules 106 to generate additional data for a user or medical practitioner. Third party integrations 108 may include third party services 160, third-party monitoring services 162, third party data services 164, mobile device 166, and apps 168.

The third-party monitoring services 162 and/or data services 164 may be provided indirectly to the wearable device 102 via apps 168 that may interact with sensor data and device 102. Third party services 160 may provide monitoring services 162 and/or data services 164 to device 102 via a Wi-Fi network, a cellular network, cloud networks, or the like. The third-party monitoring services 162 and/or data services 164 may be provided directly to the device 102 or indirectly to device 102 via a mobile device 166.

In some implementations, the data processing modules 106 and algorithms 140 described herein may be executed on wearable device 102. In some implementations, the data processing modules 106 and algorithms 140 described herein may be executed on the system 100. In some implementations, the data processing modules 106 and algorithms 140 described herein may be executed on environment 101. In some implementations, the data processing modules 106 and algorithms 140 described herein may be executed on computing devices (e.g., computing devices 107) or third-party integrations (e.g., third-party integrations 108).

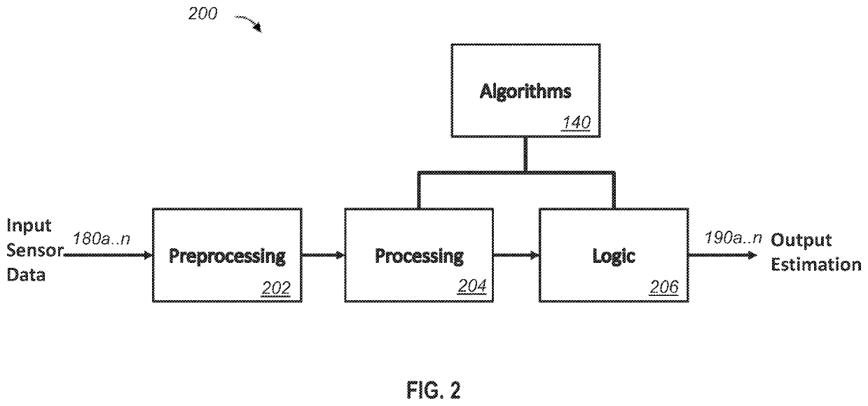

FIG. 2 illustrates a flow diagram of an example process 200 for generating biological data characterizations and/or estimates. In short, the process 200 includes obtaining sensor data corresponding to at least one of inputs 180a through 180n (e.g., 180a . . . 180n), preprocessing (202) at least a portion of the sensor data, processing (204) the preprocessed data, and performing (206) logic to characterize vasodilation activity associated with a PPG signal obtained at one or more skin surface sites. In some implementations, the processing block 204 and/or logic block 206 may include any or all steps associated with algorithms 140. The process 200 may use one or more sensor inputs 180a . . . n to perform operations using memory 118 and processors 114 of the wearable device 102, and/or memory 115 and processors 114 of the computing device 107.

For example, an optical sensor input of sensor 104b may be received as input 180b into preprocessing block 202. The preprocessing block 202 may perform signal preparation, signal normalization, and/or noise reduction before providing a preprocessed signal to processing block 204. Processing block 204 may perform Kalman filtering, harmonic mean analysis, and/or other smoothing and/or noise removing techniques. A processed signal may be provided to logic block 206 to undergo comparisons, analysis, and further signal analysis. In some implementations, the processing block 204 and the logic block 206 may function to iteratively pass data back and forth to further smooth and curate the signal. A final vasodilation activity estimate may be generated by logic block 206 upon meeting predefined criteria described in detail throughout this disclosure and in at least PCT Application PCT/US2022/071701, incorporated herein in its entirety.

### Systems for Characterizing Biological Indication(S)

FIG. 3 illustrates a first wearable device 102a and a second wearable device 102b for measuring biological data and response asymmetry across a right and left limb, respectively. In this example, the device 102a is worn on a left limb 302 of a body of a person and the device 102b is worn on a right limb 304 of the body of the person. In some implementations, the system 100 described herein includes a first system or device 102 (e.g., wearable device 102a) positioned on a left limb or appendage (e.g., arm, leg, finger, hand, foot, toe, ear, etc.) of a person and a second system or device 102 (e.g., wearable device 102b) positioned on a right limb or appendage (e.g., arm, leg, finger, hand, foot, toe, ear, etc.) of the person. The first and second devices 102a, 102b may measure similar parameters or features so that the parameters or features are comparable over time and/or on an event-by-event basis to detect biological data, asymmetrical biologic responses, and/or deviation from a baseline (e.g., individualized or population based). For example, a hardware processor as part of the system 100 or communicatively coupled to devices, such as laptop 306 and/or mobile computing device 308 may be configured to compare right side blood volume signals (e.g., in response to an application of heat) to left side blood volume signals (e.g., in response to application of heat) to determine whether an anomalous biologic event has occurred. In some implementations, the application of heat may be applied to both devices 102a, 102b to determine biological data that may later be analyzed to generate predictions, estimates, or treatment recommendations. Further, a method performed by the hardware processor may include obtaining PPG signals that may be used to generate increasingly accurate estimates of vasodilation activity for a particular skin surface site. Such activity may be assessed across a left and a right limb, for example to compare the activity across limbs and determine whether particular left and right estimates may indicate dysfunction or the occurrence of an anomalous biologic event pertaining to vasodilation, blood pressure, arterial pressure, or the like.

### Example Wearable Device

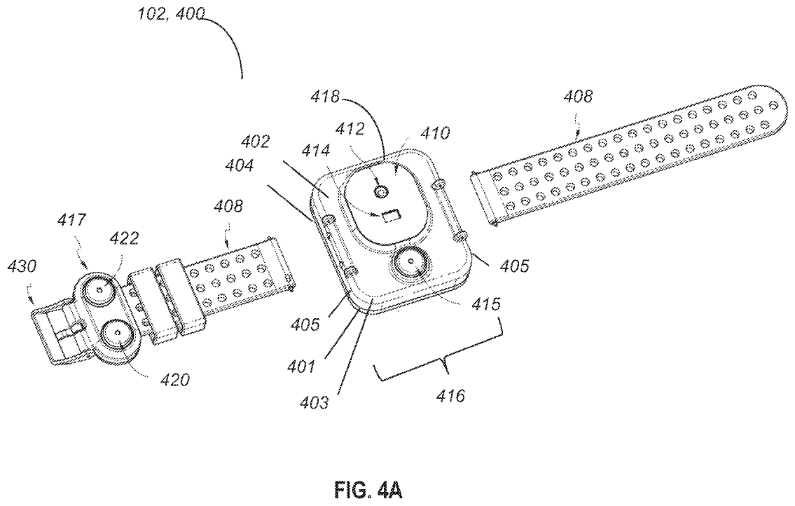

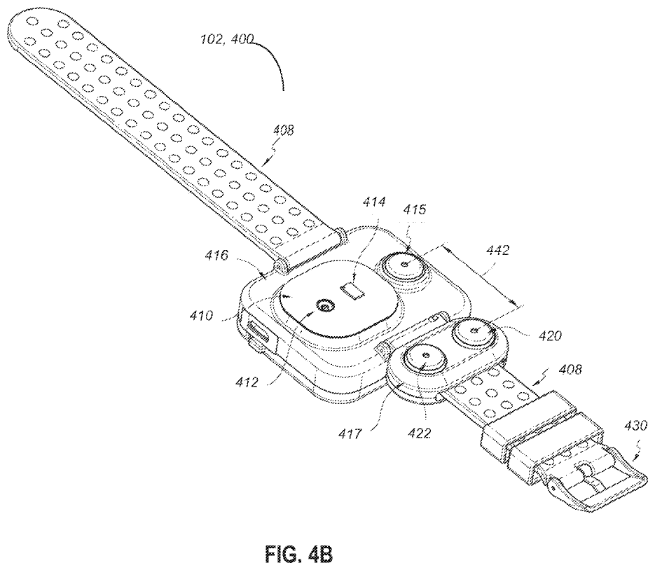

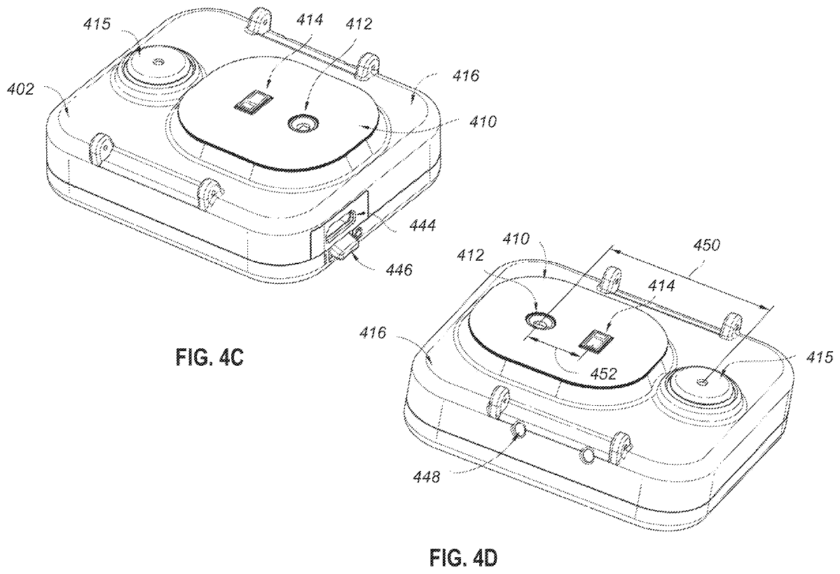

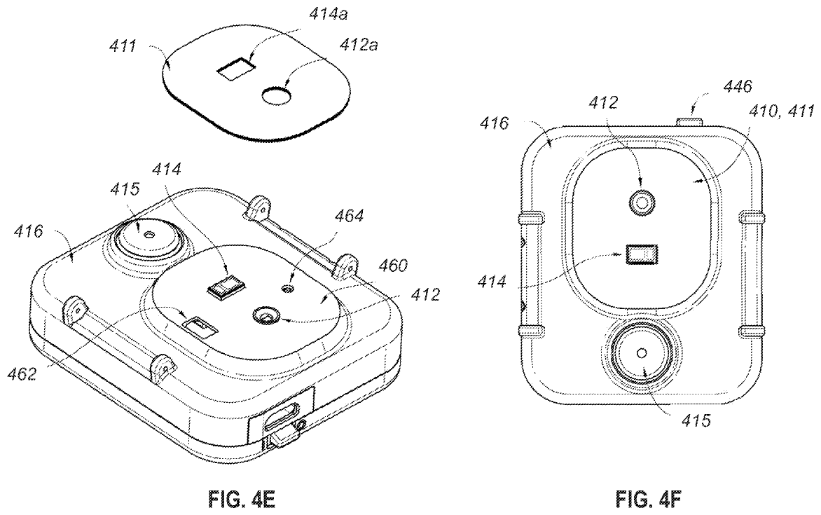

FIGS. 4A-4F illustrate a skin-facing side of a system or device 102, including a surface of a body (e.g., limb 302 or limb 304). The device 102 may represent the system 100 on the device 102. The device 102 may be configured to detect an anomalous biologic event, biologic signals, and/or related data by providing heat to a skin surface and measuring a vasodilation response of the skin surface. The system or device 102 may further function to measure one or more additional parameters, biologic signals, etc. as will be described in greater detail elsewhere herein. In some implementations, the device 102 may use a measured bioimpedance (BioZ) to validate or invalidate a vasodilation measurement from another sensor on device 400.

As shown in FIG. 4A, the device 102 includes a body portion 416 having a housing 401 which includes a first surface 404 opposite a second surface 402 in contact with a skin surface of a person. The first surface 404 and second surface 402 may be coupled via one or more or a plurality of sidewalls 405. The first surface 404, second surface 402, and plurality of sidewalls 405 may define an interior 403 (not shown). For example, one or more sidewalls 405 may extend from a perimeter of the first surface 404 and couple to a perimeter of the second surface 402. The first surface 404 and/or second surface 402 may include one or more sensors positioned thereon. For example, one or more sensors on the first surface 404 may measure an environment of the person wearing or using the wearable device 102, and one or more sensors on the second surface 402 may measure one or more properties, features, or characteristics of the skin surface of the person. Alternatively, the first surface 404 may include one or more sensors or imagers or cameras for assessing a facial region of a person.

The wearable device 102 may include a heat source 410 (e.g., stimulus source 132) in communication with the skin surface. The heat source 410 may be configured to heat the skin surface to a target temperature or a pre-determined temperature. The heat source 410 may be a heating element; thin film resistance flexible heater; polyimide heater; optical heater (e.g., a laser), etc. In other implementations, the heat source 410 an environmental heat source, for example a warm room, a warm enclosure, or other warm environment. In such implementations, the stimulus may be a change in environmental temperature, for example, from a warm environment to a cool environment or a cool environment to a warm environment. In some implementations, a heat source 410 is positioned on a second surface 402 of the body portion 416, so that there is coupling or contact between the heat source 410 and a skin surface. Alternatively or additionally, a heat source 410 or one or more sensors (e.g., sensors 412, 414) and/or electrodes (e.g., electrode 415, 420, 422) may be positioned on a band 408 of the device 102, such that the body portion 416 is separate from a sensor module 418 (e.g., including one or more sensors 104, 412, 414, etc.) that includes the heat source 410 and the one or more sensors selected from sensors 104, for example.

The band 408 may be an adjustable or tensionable band including a buckle 430 on a first side and a series of receiving notches on a second, opposing side. Band 408 may allow for similar or alternative number and arrangements of electrodes 415, 420, and/or 422. In some implementations, a Velcro® or another hook-and-loop fastener (not shown), and/or a stretchable material (e.g., silicone, rubber, Lycra, Spandex, Elastane, neoprene, leather, fabric, etc.) can be employed along at least a portion of the band 408.

In some implementations, the band 408 may include connectors (not shown) for connecting electrodes (for example, electrodes 420 and 422) to other elements housed in the body 416 including, but not limited to, a power supply (for example, a battery) and a processor (for example, processor(s) 114 of FIG. 1B). The connectors can be electrical traces, for example wires, conductive ink, circuitry or another connector, optical connectors, for example fiber optic cable, or other suitable connector that may be on, or at least partially embedded in a band 408. In some implementations, these traces from the electrodes 415, 420, and/or 422 of the band 408 to elements of the body 416 may be formed by cold molding or insert molding. In some implementations, connector wires may be threaded, woven, or sewn into the material of the band 408 and/or holes, channels, or other apertures in the band 408. In some implementations, the band 408 is at least partially made of a conductive material. In some implementations, connectors from a band 408 are connected to components (e.g., a battery, processor(s) 114) inside the body 416 through one or more holes (e.g., holes 448 of FIG. 4D) in the body 416.

The band 408 may carry an electrode housing 417 that may hold additional electrodes, such as two electrodes 420 and 422. The electrode housing 417 may be integral with or formed as a unitary structure with the buckle 430. In some implementations, the electrode housing 417 may be openable and/or removable from the band 408. The electrode housing 417 may include a lower portion and upper portion for housing one or more electrodes, for example electrodes 420 and 422. The lower portion and/or upper portion may include features for securely seating and retaining electrodes and/or an end of band 408 and/or a buckle 430 and/or an upper portion of the band. These securing features can include recesses, notches, alignment pins, fasteners, and/or clips to hold and/or align the assembly components. In some implementations, the electrode housing 417 may be secured to a particular location on the band 408, where the electrode housing 417 cannot slide (for example, with pins and holes 448). In some implementations, the lower portion and upper portion securely mate together and cannot be opened. In some implementations, the lower portion and upper portion securely mate together and can be opened by manipulating the pieces, for example, by sliding or twisting the pieces against each other, and/or opening a latch, button, or other retention feature. In some implementations, the electrode housing 417 is secured to the band 408 and can slide along the length of the band. For example, the lower portion and upper portion may secure together over and/or around the band 408 and provide a clamping force to the band 408 to secure the electrode housing 417 in position while being slidable along the band 408.

Referring again to FIG. 4A, the body portion 416 further includes a blood volume sensor 412 and a skin temperature sensor 414. The blood volume sensor 412 can be integrated into a form factor such as the device 400 that improves continuous anomalous cardiac event monitoring. The blood volume sensor 412 can measure parameters that can provide vasodilation response. The skin temperature sensor 414 can also be integrated into the device 102 or system 100. The skin temperature sensor 414 may be positioned on the second surface 402 and configured to measure a temperature of the skin surface in contact with the heat source 410. The blood volume sensor 412 may be positioned on the second surface 402 and configured to measure a blood volume of the skin surface. The blood volume sensor may be a photoplethysmography sensor or an impedance plethysmographic sensor. The blood volume sensor may employ light at one or more of 500-570 nm (green), 610-760 nm (red), 450-500 nm (blue), or infra-red (>760 nm) wavelength, or a combination thereof. Different wavelengths may be more appropriate for different applications, for example green (500-570 nm) light may be more accurate for heart rate measurements (e.g., heart rate variability, heart rate, etc.). In addition to, or alternatively, the blood volume sensor 412 may also measure one or more of: heart rate, heart rate variability, or oxygen saturation.

FIG. 4B illustrates a perspective view of the wearable device of FIG. 4A. A second surface 402 of a body 416 is depicted. The second surface 402 of the body 416 may be similar or identical to a second surface 402 of other devices described herein, for example second surface 402 may include a heat source 410, blood volume sensor 412, and skin temperature sensor 414. In some implementations, the second surface 402 includes one electrode 415. A band 408 with a buckle 430 may include an electrode housing 417 carrying additional electrodes, such as two electrodes 420 and 422. The electrodes 420 and 422 (and/or other additional electrodes and/or sensors) may be positioned on the second surface 402 and/or a tensionable band 408 of the device 102. The electrodes 415, 420, and/or 422 may be located away from the surface of the device 102. For example, electrode 415 may be placed on a raised platform 460 of the second surface 402 of the body 416. As discussed above with regard to electrodermal sensors, sensors 412 and 414 and/or electrodes 415, 420, and/or 422 may be spaced at preselected distances. In some implementations electrodes 420 and 422 are spaced at a distance of about 5 mm to about 100 mm, for example 5 mm to about 10 mm, about 10 mm to about 20 mm, about 20 mm to about 30 mm, about 30 mm to about 40 mm, about 40 mm to about 50 mm, about 50 mm to about 60 mm, about 60 mm to about 70 mm, about 70 mm to about 80 mm, about 80 mm to about 90 mm, about 90 mm to about 100 mm, measured from a center point of each electrode 420, 422.

In some implementations, electrodes 420 and 422 may be spaced apart from the heat source 410 and/or electrode 415 by a distance 442, which can be about 40 mm to about 300 mm, for example about 40 mm to about 50 mm, about 50 mm to about 60 mm, about 60 mm to about 80 mm, about 80 mm to about 100 mm, about 100 mm to about 120 mm, about 120 mm to about 150 mm, about 150 mm to about 175 mm, about 175 mm to about 200 mm, about 200 to about 225 mm, about 225 mm to about 250 mm, about 250 mm to about 275 mm, about 275 mm to about 300 mm, measured from a center point of the electrodes 420, 422 to a center point of the heat source 410 and/or the electrode 415. Still further, electrode 415 may be spaced apart from a heat source 410 by a distance which can be about 10 mm to about 20 mm, about 20 mm to about 30 mm, about 30 mm to about 40 mm, about 40 mm to about 50 mm, about 50 mm to about 60 mm, about 60 mm to about 70 mm, about 70 mm to about 80 mm, about 80 mm to about 90 mm, about 90 mm to about 100 mm, measured from a center point of the electrode 415 and a center point of the heat source 410. When the body 416 is secured to the wrist with the band 408, in some implementations, the electrodes 420 and 422 are positioned on the band such that they contact the skin approximately along a midline of palm side of the wrist. The electrode 415 may be positioned approximately on a midline on the dorsal side of the wrist.

FIG. 4C illustrates a partial view of the wearable device 102 of FIG. 4A. As shown, the body portion 416 of the device 102 includes a port 444 for electrically coupling the device 102 to a power source, for example to charge a battery (such as battery power 133) in the device 102. Additionally or alternatively, port 444 may electrically couple the wearable device to an external or remote computing device (e.g., laptop, desktop, server, workstation, etc.) to download data from the device or upload system parameters or install updates to the wearable device 102. Port 444 may also be used to connect auxiliary sensors, an input/output device (keyboard, joystick, buttons, switches, printer, camera, display), and/or memory unit. The wearable device 102 may further include one or more user interface elements 446, for example one or more buttons and/or switches, that may be used for example to power on and off the device, to input user specific reactions, features, or characteristics, to customize an interface or functionality of the user device, to mark events, to initiate pairing or data transfer, to call for help, etc. User interface elements 446 may alternatively or additionally include output and/or feedback elements, such as a speaker, light, and/or haptic stimulator. In some implementations, user interface element 446 may be used, for example, to indicate power on, charging, low battery, pairing mode, heating phase, malfunction, health event, and/or other status of the device 400 and/or user. In some implementations, user interface element 446 may be a feedback element that includes one or more LED behind a smoked, translucent, or transparent window. In some implementations, there is no display screen on the body 416.

FIG. 4D illustrates a rotated view of the wearable device 102 of FIG. 4C. As shown, the second surface 402 of body 416 may include a heat source 410, blood volume sensor 412, skin temperature sensor 414, and electrode 415, as discussed above. The second surface 402 of body 416 may be arranged to contact the back (dorsal) side of the wrist when worn to locate the heat source 410, blood volume sensor 412, skin temperature sensor 414, and electrode 415 generally along a midline of the wrist. Blood volume sensor 412 and skin temperature sensor 414 may be placed within the heat source 410 spaced at a distance 452 from each other. Distance 452 may be about 10 mm to about 100 mm, for example 10 mm to about 20 mm, about 20 mm to about 30 mm, about 30 mm to about 40 mm, about 40 mm to about 50 mm, about 50 mm to about 60 mm, about 60 mm to about 70 mm, about 70 mm to about 80 mm, about 80 mm to about 90 mm, about 90 mm to about 100 mm, measured from a center point of the blood volume sensor 412 and a center point of the skin temperature sensor 414. Similarly, blood volume sensor 412 may be placed at a distance 450 from the electrode 415. Distance 450 may be about 10 mm to about 200 mm, for example 10 mm to about 20 mm, about 20 mm to about 30 mm, about 30 mm to about 40 mm, about 40 mm to about 50 mm, about 50 mm to about 60 mm, about 60 mm to about 70 mm, about 70 mm to about 80 mm, about 80 mm to about 90 mm, about 90 mm to about 100 mm, about 100 mm to about 120 mm, about 120 mm to about 140 mm, about 140 mm to about 160 mm, about 160 mm to about 180 mm, about 180 mm to about 200 mm, measured from a center point of the blood volume sensor 412 and a center point of the electrode 415.

FIG. 4E illustrates a perspective view and an exploded view of the wearable device 102 of FIG. 4C. As shown, the second surface 402 of the body 416 may include a raised platform 460. Platform 460 may include the heat source 410, blood volume sensor 412, and skin temperature sensor 414. In some implementations, the platform 460 may include electrode 415. Platform 460 may improve contact between the skin and the heat source 410, blood volume sensor 412, and skin temperature sensor 414. In some implementations, the platform 460 is sized to cover or substantially cover the back of the wrist when device 102 is worn. In some implementations, the platform 460 is flexible and/or shaped, for example, curved, and may increase a contact area between the platform 460 and the skin when the device 102 is worn. In some implementations, heat source 410 symmetrically surrounds the blood volume sensor 412 and/or symmetrically surrounds the skin temperature sensor 414. In some implementations, heat source 410 may surround the blood volume sensor 412 with an area approximately equal to the area of heat source 410 that surrounds the skin temperature sensor 414.

In some implementations, the heat source 410 may include a warming plate 411, as shown in FIG. 4E. For example, the heat source 410 may include a warming plate 411 for increased heat distribution and/or heat retention. The second surface 402 of body 416 may include an opening, such as opening 462, to allow the heat source 410 and/or warming plate 411 to communicate with and/or access other components inside the body 416, for example the processor(s) 114 and battery power 133. The second surface 402 of the body 416 may include a thermistor 464 or other temperature sensor for monitoring the temperature of the heat source 410 and/or warming plate 411. The thermistor 464 may provide a heater temperature measurement, which may be used to control the heat source 410.

Warming plate 411 may include apertures for components surrounded and/or enclosed by the heat source 410, for example aperture 412a for blood volume sensor 412 and aperture 414a for skin temperature sensor 414. Aperture 412a and aperture 414a may allow improved contact between the skin and blood volume sensor 412 and skin temperature sensor 414. In some implementations, the blood volume sensor 412 includes two separate components, for example an emitter and a detector, and accordingly aperture 412a would include an aperture for each component. In general, heat source 410 may be a layered or laminate structure. In some implementations, the heat source 410 may include dimples and/or perforations for heat distribution and/or dissipation. Perforations may also improve adhesion.

FIG. 4F illustrates a front view of the wearable device 102 of FIG. 4C. The warming plate 411 is shown installed on heat source 410 of wearable device 102. Alternative or additional features, such as ridges, channels, fins, and the like may also be included on a heat source 410 to improve uniform heating and cooling and/or direct heating and cooling. For example, surface features may be used to direct heat from the heat source 410 toward or away from an inner wrist-facing side of the device 102. The surface features may alternatively or additionally be located on a warming plate 411. Heat source 410 may be positioned on a raised platform 460 of the second surface 402 of the body 416. In some implementations, the heat source 410 is arranged farther from the hand than the electrode 415 when the device 102 is worn on the wrist (or other appendage).

### Safety Systems

As mentioned above, monitoring for stroke symptoms and/or physiological symptoms using a stimulus source contacting a skin surface of a user can present some risks should the stimulus source suffer a failure. Failures during warming stimulus cycles may lead to bodily injury (e.g., burns) as the stimulus source approaches harmful temperatures. Typical safety monitoring systems having single safety mechanisms or software only based safety systems may experience single fault failures, resulting in the inability to detect abnormalities. In this case, failure of the safety monitoring systems may result in harm to the user. To improve overall safety, the safety system may include double-fault protection by having two or more safety components, as well as redundant components, positioned in series to maximize effectiveness. Thus, if one of the safety components fails, an additional safety features can continue to monitor for unsafe conditions. For example, if a first safety component fails, when a second safety component of the safety system senses a dangerous condition, the safety system may reduce and/or cutoff the voltage from a power source to a thermal stimulus such that the device will not function. As stated in ISO Standard IEC60604, medical devices should continue to safely function in the case of a single-fault failure.

In operation, the safety system devices and systems described herein may, under normal operating condition when the device is obtaining physiological data from a skin surface site of a person to estimate vasomotor responses (e.g., vasodilation activities, responses, levels, signals, parameters, time-based parameters, etc. and/or vasoconstrictive activities, responses, levels, signals, parameters, time-based parameters, etc.) of the person, transmit an activation signal to a heating stimulus and a power source. In response to one or more fault conditions in either a temperature monitoring system or a hardware circuit, the safety system may transmit a cutoff signal to disconnect the stimulus source from the power source.

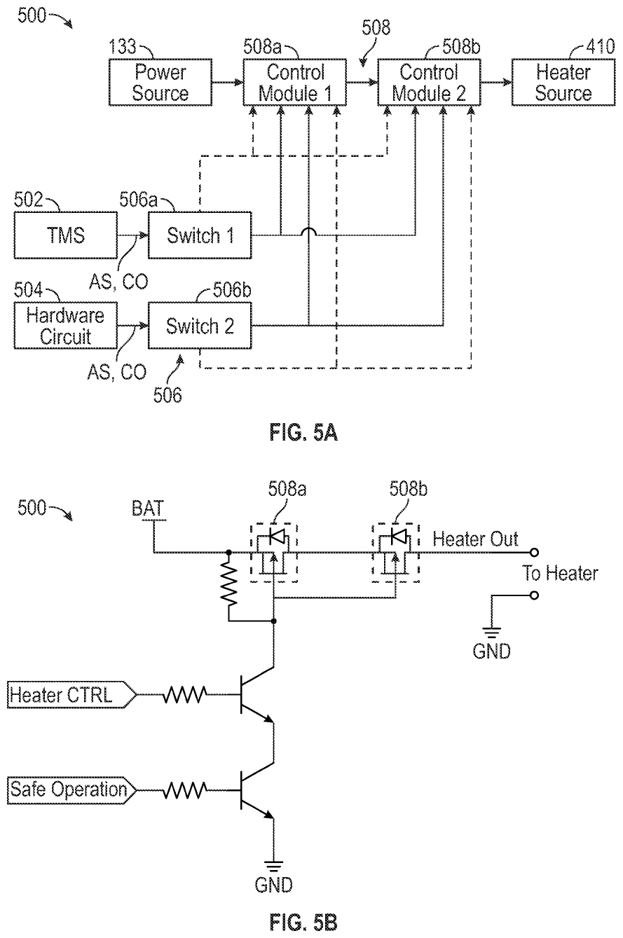

FIG. 5A illustrates a non-limiting schematic diagram of a temperature safety system 500 which may be integrated into the body portion 416 of the wearable device 400 or into a separate component of the wearable device 400 to manage power to a stimulus source, such as heat source 410, for example. The safety system 500 may prevent harm and/or injuries, such as tissue injuries, to a user in the case of malfunctions, such as software and/or hardware malfunctions, during a stimulus cycle. The safety system 500 may include a temperature monitoring system (TMS) 502 and/or a hardware electronic circuit 504 to monitor one or more temperatures during said stimulus cycle. For example, in some implementations, the monitored temperatures may be related to the heat source 410 performing as the stimulus source and/or the skin surface site of the user being acted on by heat source 410. The safety system 500 may control the power supplied from power source 133 to the heat source 410. Firmware and/or other processes may monitor the voltage from the power source 133 to the heat source 410 to maintain consistent power output as the power level of the power source 133 changes. The safety system 500 may transmit an activation signal AS when the monitored temperatures satisfy a threshold. Once the monitored temperatures cross the threshold temperature, based at least upon either the TMS 502 or the hardware electronic circuit 504 determining that one or the monitored temperature has reached and/or crossed the predefined threshold temperature, the safety system 500 may transmit a cutoff signal CO to interrupt the power supplied to the heat source 410 from the power source 133. In some implementations, a lack of a signal from the monitored temperatures corresponding to either the TMS 502 and/or the hardware electronic circuit 504 may also output a cutoff signal CO. In some implementations, the TMS 502 and/or the hardware electronic circuit 504 may output a cutoff signal CO to one or more switches 506 to disrupt the voltage between the power source 133 and the heat source 410. The one or more switches 506 may include an NPN transistor, a PNP transistor, or the like performing as a switch and/or amplifier.

In some implementations, the TMS 502 may be in communication with the skin temperature sensor 414, mentioned above, to monitor the skin surface site responsive to the stimulus source (e.g., heat, cold,) applied to said skin surface site during the stimulus cycle. The TMS 502 may also include a proportional integral derivative (PID) controller or the like. In some implementations, the PID controller may drive the heat source 410 “on” or “off”. When “on,” the battery power is dissipated across the heat source 410. The PID controller may receive control inputs from the stimulus cycle settings, the inputs specifying the speed of which the heat source 410 temperature increase, as well as the duration of the “on” and “off” periods. The stimulus cycle may also include heating the skin surface site to a target temperature. During operation, the TMS 502 can receive a first temperature reading T_{S} from the skin temperature sensor 414 corresponding to the temperature of the skin surface site associated with the wearable device 400. The TMS 502 can then output a signal to continue or terminate the stimulus cycle at least based on the skin surface temperature reading. In some implementations, the TMS 502 may continuously monitor the temperature of the skin surface over time. In some implementations, the TMS 502 may monitor the first temperature reading T_{S} of the skin surface site according to a schedule and/or at set intervals. The TMS 502 may output a cutoff signal CO to a first switch 506a to disrupt the voltage between the power source 133 and the heat source 410, once the target temperature is reached, to interrupt the voltage between the power source 133 and heat source 410. In some implementations, the heat source 410 may be cycled on and off once the target temperature is reached in order to maintain said target temperature. The TMS 502 may also trigger a cutoff signal CO to switch 506a to interrupt the voltage between the power source 133 and heat source 410 in response to determining that the first temperature reading T_{S} has reached and/or exceeded a first temperature threshold T₁. For example, the TMS 502 may be set to interrupt the voltage when the temperature T_{S} at the skin temperature site is greater than 50 degrees Celsius, greater than 49 degrees Celsius, greater than 48 degrees Celsius, greater than 47 degrees Celsius, greater than 46 degrees Celsius, greater than 45 degrees Celsius, greater than 44 degrees Celsius, greater than 43 degrees Celsius, greater than 42 degrees Celsius, greater than 41 degrees Celsius, or greater than 40 degrees Celsius. In some implementations, the one or more processors 114 may also interrupt the voltage if the heat source 410 is powered for longer than a set amount of time. In some implementations, the TMS 502 may be set to interrupt the voltage when the temperature T_{S} at the skin temperature site reaches a maximum value of 44 degrees Celsius. In some implementation, the TMS 502 may be set to interrupt the voltage when the temperature T_{S} at the skin temperature site reaches a value of approximately 42 degrees Celsius as the vasodilation response is no longer affected by the heat source 410. In some implementations, the TMS 502 may interrupt the voltage between the heat source 410 and the power source 133 if the power on time for the heat source 410 exceeds 240 seconds, exceeds 180 seconds, exceeds 120 seconds, exceeds 90 seconds, exceeds 60 seconds, exceeds 30 seconds, or exceeds 15 seconds. In some implementations, the TMS 502 may monitor the signals of the first temperature reading T_{S} to determine whether the wearable device 400 is in contact with the skin surface of the person by determining if a skin temperature is found. If not, the TMS 502 may output a signal to the heat source 410 to terminate the stimulus cycle at least based on the not found skin temperature reading. The TMS 502 may be implemented with the hardware components described above with respect to FIGS. 1A to 4F. The TMS may include programmed instructions for performing processes as discussed herein for monitoring temperature and controlling the stimulus source.

The hardware electronic circuit 504 may also continuously and/or intermittently monitor the second temperature reading Tr corresponding to the heat source 410 over time. In some implementation, as the temperature of the heat source 410 increases, the hardware electronic circuit 504 may trigger a cutoff signal CO to a second switch 506b to interrupt the voltage between the power source 133 and heat source 410 in response to the second temperature reading TT of the heat source 410 reaching and/or exceeding a second temperature threshold T₂. For example, the hardware electronic circuit 504 may interrupt the voltage between the power source 133 and heat source 410 when the second temperature reading T₂ is greater than 50 degrees Celsius, greater than 49 degrees Celsius, greater than 48 degrees Celsius, greater than 47 degrees Celsius, greater than 46 degrees Celsius, greater than 45 degrees Celsius, greater than 44 degrees Celsius, greater than 43 degrees Celsius, greater than 42 degrees Celsius, greater than 41 degrees Celsius, or greater than 40 degrees Celsius. In some implementations, the hardware electronic circuit 504 may trigger a cutoff signal CO to a second switch 506b to interrupt the voltage between the power source 133 and heat source 410 in response to the heat source 410 reaching and/or exceeding a maximum second temperature threshold T₂ at 48 degrees Celsius. In some implementations, the threshold temperature T₂ may be lowered to trigger the cutoff signal CO at a lower temperature if the safety system 500 does not include other safety circuits (e.g., TMS 502). Once the hardware electronic circuit 504 determines that the heat source 410 has reached a temperature below the second temperature threshold T₂, the cutoff signal CO may end, allowing the voltage between the power source 133 and heat source 410 to flow.

In some implementations, the hardware electronic circuit 504 of the temperature safety system 500 may include the thermistor 464 for monitoring the second temperature reading Tr corresponding to the heat source 410, which may be used to control the heat source 410. In some implementations, the resistance of the thermistor 464 may also increase as the temperature of the heat source 410 increases. The rise in temperature may trigger the cutoff signal CO to switch 506b to interrupt the voltage between the power source 133 and heat source 410 in response to the heat source 410 reaching and/or exceeding the second temperature threshold T₂.

In some implementations, the temperature safety system 500 can further include one or more control modules 508 to provide redundancy protection for interrupting the voltage between the power source 133 and heat source 410 in the case of a malfunction. In some implementations, the one or more control modules 508 are in series. The one or more control modules 508 may also include field effect transistors. The one or more control modules 508 may be in communication with the one or more switches 506, such as first switch 506a and/or the second switch 506b. The one or more control modules 508 may act like as a backup switch controlled by at least Boolean logic and may receive an input from at least one of the one or more switches 506. In some implementations, the first switch 506a and second switch 506b can jointly and/or simultaneously transfer an activation signal AS and/or a cutoff signal CO to control module 508a and control module 508b (shown in solid lines). In other implementations, the first switch 506a and second switch 506b may independently and/or separately transfer the activation signal AS and/or a cutoff signal CO to control module 508a and control module 508b (shown in dotted lines). Depending on the transferred signal, both control module 508a and control module 508b may open or close providing redundant protection in case any of the one or more control modules 508 fail.

In some implementations, the safety system 500 may prevent the heat source 410 from reaching a temperature above a threshold temperature by included additional components in the temperature safety system 500. For example, one or more resistors may be arranged in such a configuration to limit the voltage to the heat source 410. The one or more resistors positioned along the path between the power source 133 and the heat source 410 may be in parallel to decrease the voltage such that the voltage does not warm the heat source above a threshold temperature. In some implementations, the safety system 500 may include a step-down transformer (also mentioned herein as a “voltage converter”) positioned between the power source 133 and the heat source 410. The step-down transformer can reduce the voltage from the power source 133 to a lower voltage such that the heat source 410 may not receive sufficient power to reach above a threshold temperature.

FIG. 5B illustrates an exemplary implementation of the temperature safety system 500 as described above. In some embodiments, the system can include a variety of different resistors that can correspond to different ohmic values, such as 1 kΩ (2 or 4.7 kΩ or other values.

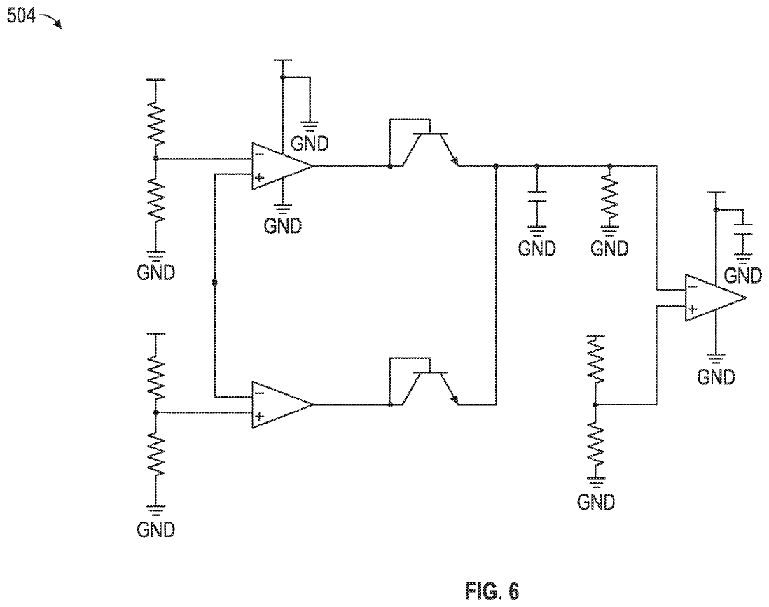

FIG. 6 illustrates an exemplary implementation of the hardware electronic circuit 504 as described above.

### Stimulus Cycle Examples

Stimulus cycles may be customized to correspond to time constraints and other paraments. For example, for monitoring a user at a hospital and/or at home, longer durations may be chosen to capture physiological signals. For shorter time periods, such as in a mobile stroke unit, the stimulus cycle may be curated to complete more stimulus cycles in a short amount of time.

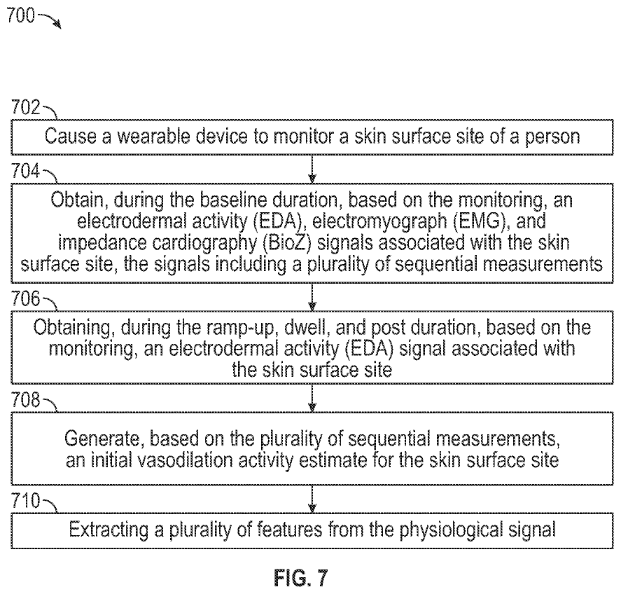

FIG. 7 illustrates a flow diagram of an example warming process 700 for estimating vasodilation activity at least based on one or more vasodilation estimates. In general, the process 700 utilizes the systems 100, 101 and algorithms 140 described herein to enable the wearable device 102 to obtain physiological signals (e.g., electrodermal activity (EDA), electromyograph (EMG), and impedance cardiography (BioZ) and compute estimated vasodilation activity for a person wearing the device 102. The process 700 may be performed by a processor (e.g., processor 114) and memory (e.g., memory 118) of the wearable device 102 to execute computer-readable instructions as described in detail throughout this disclosure.

At block 702, the process 700 includes causing a wearable device to monitor a skin surface site of a person. The monitoring may be carried out by monitoring engine 146 and/or third-party integrations 108. The wearable device (e.g., wearable device 102) may be worn by the person and may be placed in contact with or near to) the skin surface site. A heating stimulus (e.g., stimulus source 132) may trigger changes in temperature to occur at the skin surface site. The changes may be sensed by any number of sensors 104. The monitoring may be performed over time and may be performed according to predefined stimulus application cycles. The stimulus cycle may be separated into one or more electrode phases to obtain physiological signals. For example, the stimulus cycle may be separated into phases measuring EDA, phases measuring EMG, and/or phases measuring BioZ. A baseline duration may be comprised of, for example, a first phase measuring EDA, a phase measuring EMG, and a phase measuring BioZ. A ramp-up, dwell, and post phase may be comprised of a second EDA measuring phase. During the ramp-up, dwell, and post phase, the stimulus source may generate a response from the skin site for the duration of the phase. For shorter and/or time constrained stimulus cycles, such as measurements taken by a mobile stroke unit, the stimulus cycle may be comprised of a baseline duration comprised of an EMG measuring phase and a BioZ measuring phase and a ramp-up, dwell, and post phase comprised of an EDA measuring phase. The stimulus cycle may be performed once, repeated for a set number of intervals, and/or repeated continuously.

At block 704, the process 700 includes obtaining during the baseline duration, based on the monitoring, an electrodermal activity (EDA), electromyograph (EMG), and impedance cardiography (BioZ) signals associated with the skin surface site. In some implementations, the EDA, EMG, and BioZ signals may be obtained after completion of at least one stimulus cycle generated by the stimulus source (e.g., source 132). In general, the EDA signal may include a plurality of sequential EDA measurements taken over a time period and/or stimulus cycles. In some implementations, the process 700 includes obtaining during the baseline duration, based on the monitoring, electromyograph (EMG) and impedance cardiography (BioZ) signals associated with the skin surface site.

At block 706, the process 700 includes obtaining during the ramp-up, dwell, and post duration, based on the monitoring, an electrodermal activity (EDA) signal associated with the skin surface site. In some implementations, the EDA signal may be obtained after completion of at least one stimulus cycle generated by the stimulus source (e.g., source 132). In general, the EDA signal may include a plurality of sequential EDA measurements taken over a time period and/or stimulus cycles.

At block 708, the process 700 includes generating, based on the physiological signal, a vasodilation estimate for the skin surface site. For example, if the physiological signal is an EDA signal comprising a plurality of EDA measurements, the system 100 and/or system 101 may generate the vasodilation estimate for the skin surface by generating, based on the plurality of EDA measurements, an initial vasodilation activity estimate for the skin surface site, applying, to the initial vasodilation activity estimate at a time t, a set of forward noise estimation filters to generate a first output, applying, to the initial vasodilation activity estimate at a time t−1, a set of backward noise estimation filters to generate a second output, and computing a harmonic mean between the first output and the second output.

In some embodiments, generating the vasodilation activity estimate for the skin surface site may further include adjusting for a signal lag of the first output with respect to the initial vasodilation activity estimate, adjusting for a signal lead of the second output with respect to the initial vasodilation activity estimate, and removing data outliers from the first output and the second output based on the adjustment of the signal lag of the first output and/or the adjustment of the signal lead of the second output.

At block 710, the process 700 may include extracting a plurality of features from the physiological signal. For example, the system 100 and/or system 101 may extract one or more features/metrics. In some embodiments, the features/metrics are extracted and/or obtained from a source other than the physiological signal.

In some implementations, the process 700 may be applied to a first wearable device (e.g., device 102a) and/or a second wearable device (e.g., device 102b). Both devices may be monitored by monitoring engine 146 to detect events that occur for each device 102a, 102b or to detect events (e.g., using event detector 148) that occur across devices 102a, 102b. The monitoring engine 146 may utilize the alert generator 150 and the analysis module 152 to generate alerts and perform analysis with respect to such detected events.

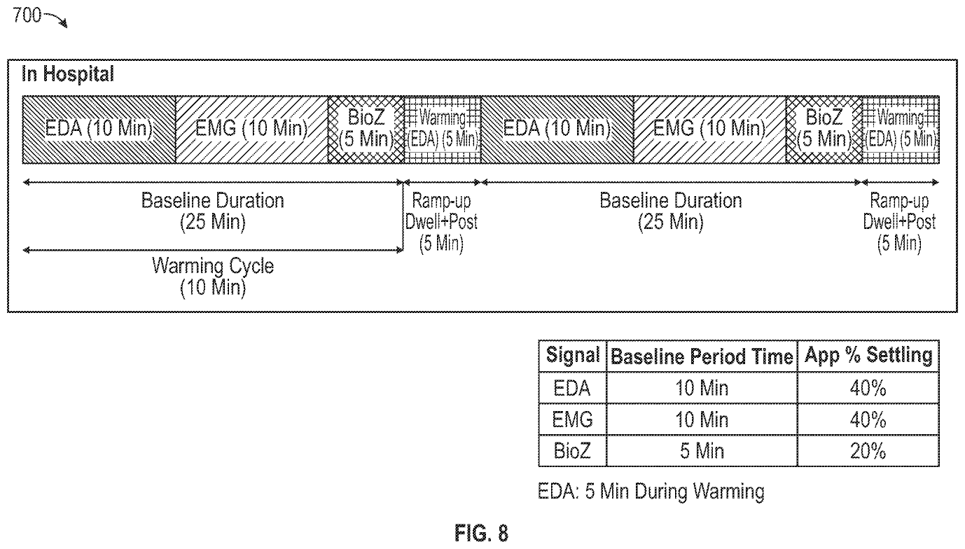

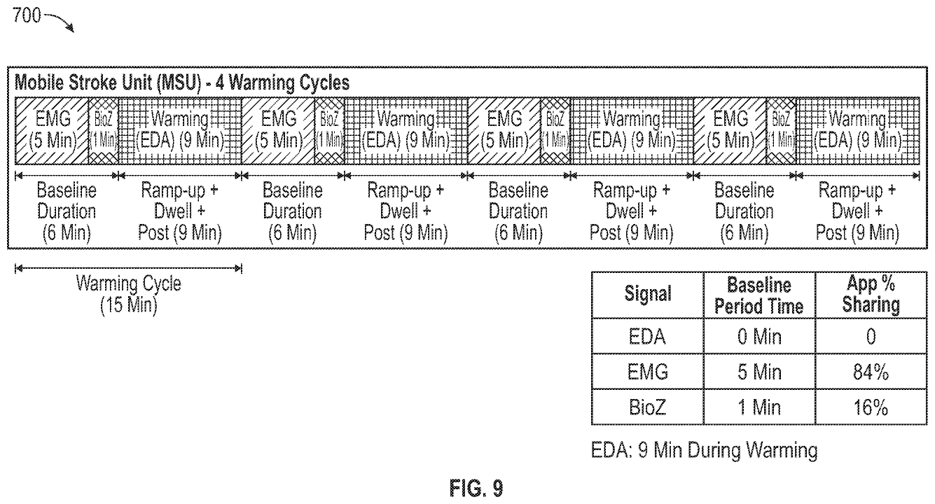

FIGS. 8 and 9 illustrate example timelines of warming process 700. FIG. 8 illustrates a thirty-minute timeline of warming process 700 which may include a twenty-five-minute baseline duration and a five-minute ramp-up, dwell, and post duration. During the baseline during, the EDA measuring phase may last ten minutes, the EMG phase may last ten minutes, and the BioZ measuring phase may last five minutes. The ramp-up, dwell, and post duration may last five minutes. The signal measuring phases contained in the baseline duration may be adjusted to occur in different orders and/or for different time periods. For example, the EMG measuring phase may occur first, followed by the EDA measuring phase, and then the BioZ measuring phase. In another non-limiting example, the duration of each signal measuring phase may also be adjusted such that the EDA measuring phase occurs for fifteen minutes, the EMG measuring phase occurs for seven minutes, and the BioZ measuring phase occurs for three minutes. The warming process 700 may be performed once or as many times as desired.

FIG. 8 illustrates an example of a fifteen-minute timeline of warming process 700 which may include a six-minute baseline duration and a nine-minute ramp-up, dwell, and post duration. During the baseline duration, no EDA measuring phase may take place, the EMG phase may last five minutes, and the BioZ measuring phase may last one minute. The ramp-up, dwell, and post duration may last nine minutes. The signal measuring phases contained in the baseline duration may be adjusted to occur in different orders and/or for different time periods. For example, the BioZ measuring phase may occur first followed by the EMG measuring phase. In another non-limiting example, the duration of each signal measuring phase may also be adjusted such that the EMG measuring phase occurs for three minutes and the BioZ measuring phase occurs for three minutes. The warming process 700 may be performed once or as many times as desired.

In the event that a second wearable device is available and in use, the process 700 may further include generating a second final vasodilation activity estimate obtained using a second wearable device with a second stimulus source placed in contact with a second skin surface site to carry out the monitoring on the second skin surface site. Further, the system 100 running on device 102b (or alternatively the system 100 communicably coupled to device 102b) may compare the final vasodilation activity estimate for the skin surface site to the second final vasodilation activity estimate of the second skin surface site and determine, based on the comparing, whether an anomalous biologic event has occurred for the person.

In some implementations, the system 100 may generate vasodilation activity estimates for purposes of determining diagnoses, dysfunction, and/or other biologic data. For example, the system 100 may generate one or more vasodilation activity estimates and provide such estimates as input into a blood pressure determination model. The system 100 may then use the model to generate a cuffless blood pressure estimate for the person based on the provided vasodilation activity estimate, and/or other metrics in the blood pressure determination model.

In some implementations, the system 100 may generate one or more vasodilation activity estimates and provides such estimates as input into an arterial stiffness model to determine stiffness metrics for a particular artery. The system 100 may use the model to generate an arterial stiffness measurement for the person based on the provided vasodilation activity estimate.

### Charging Port Safety

Charging a device while being worn by a user poses a potential health risk to said user. The continuous charging of the device while being worn increases the likelihood of burns to the user's skin. During charging periods, the temperature of a device and/or battery of the device may begin to increase to unsafe temperatures which may cause overheating and/or burns to the skin surface in contact with the device. For example, a user may experience mild to moderate burns due to overheating of a thermal stimulus and/or overcharging of a battery when the device is simultaneously worn and charging. Thus, there is a need for improved user safety to prevent burns when charging the device. Charging port(s) location, as well as placement of the device band, may also act as an additional safety system for maintaining the overall temperature of a device in conjunction with a temperature safety system.

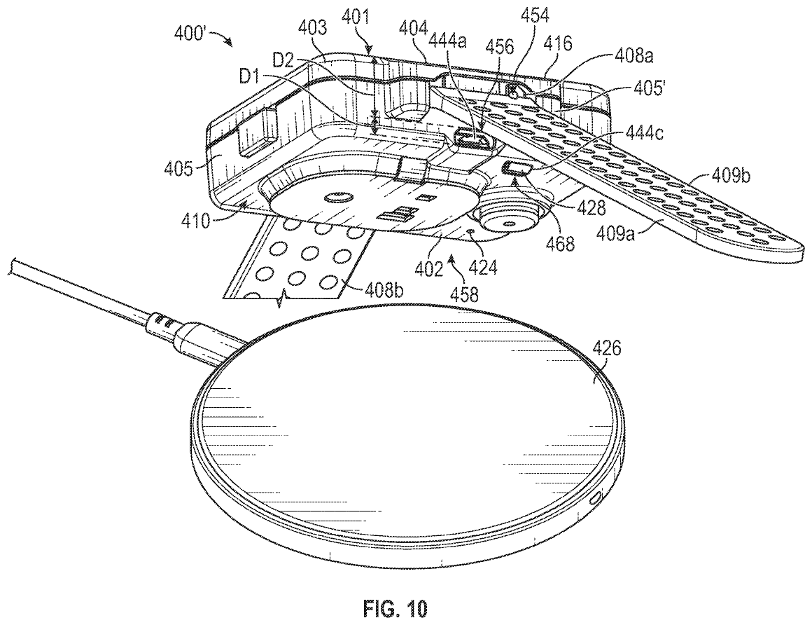

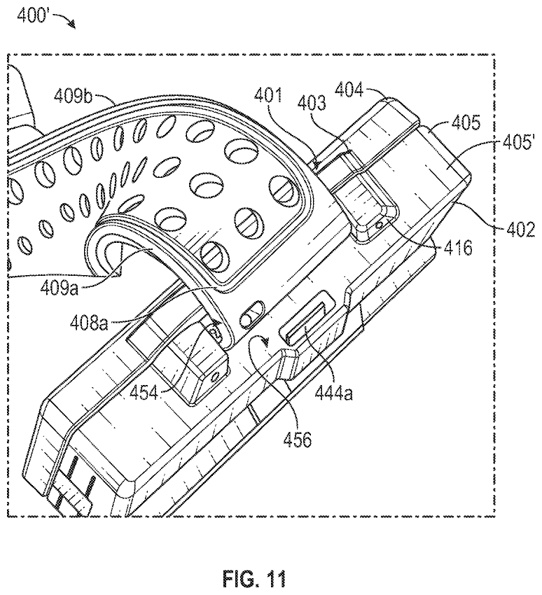

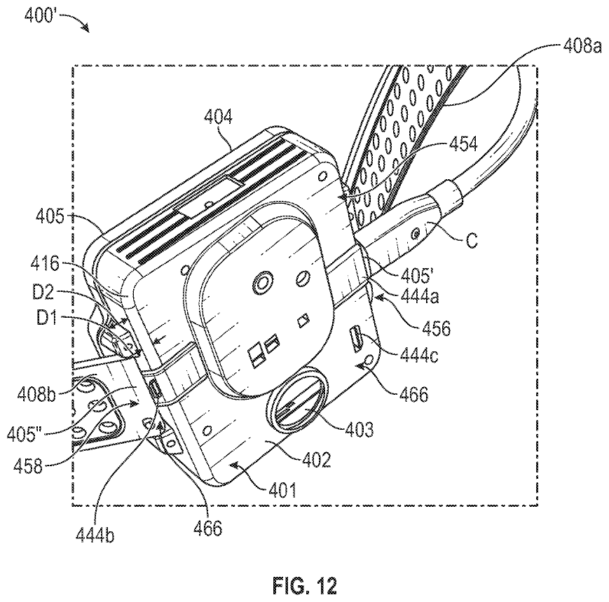

FIGS. 10-12 illustrate a partial view of an example embodiment of a wearable device 400′. FIG. 10 illustrates a perspective view of the wearable device 400′ for monitoring physiological parameters of a user. FIG. 11 illustrates another perspective view of the wearable device 400′ with first a first band 408a oriented to expose connector 444a (also mentioned herein as “connector port” and/or “charging port”) of the wearable device 400′. FIG. 12 illustrates an example of the wearable device 400′ removably connected to a cable. With reference to FIGS. 10 and 11, a Wearable device 400′ can be similar or identical to wearable device 400 described herein in some or many respects. Wearable device 400′, for example, may include housing 401, second surface 402, interior 403, first surface 404, one or more sidewalls 405, band 408, body 416 each of which is described elsewhere herein. Instead of port 444 shown in FIG. 4C, the wearable device 400′ of FIGS. 11-13 may include one or more connection ports such as ports 444a, 444b, and/or 444c.

As shown in FIGS. 10 and 11, the body portion 416 of the wearable device 400′ may include a port 444a for electrically coupling the wearable device 400′ to a power source, for example, to charge a battery (such as battery power 133) in the device 400′. In some implementations, the band 408 may be segmented to include a first band 408a and a second band 408b that couple together to secure the device 400′ to a body portion of the user. The first band 408a may connect to a first side 405′ at a first band side surface 454 and the connector port 444a may be positioned at a first connect port surface 456 on the first side 405′. The port 444a may be positioned between the first band 408a and the second surface 402 of the housing 401. For example, the port 444a may be a distance D1 from the second surface 402, which is shorter than a distance D2 from the first surface 404. Since D1 is shorter than D2, the connector port 444a may be positioned partially or entirely below the first band 408a. Band 408a thus inhibits connector port 444a from charging when the band 408a is secured around a portion of the user's body. In some implementations, the first band side surface 454 and the first connect port surface 456 may be substantially vertically aligned with one another. Additionally or alternatively, the first band side surface 454 may partially and/or fully align with the first connect port surface 456. For example, the first connector port location 456 may be positioned below the first band side surface 454 and between a first band edge 409a and a second band edge 409b of the first band 408a. The port 444a may face in a direction that intersects with the first band 408a such that the first band 408a may cover the port 444a when secured to the user. Additionally or alternatively, the first band 408a may cover the port 444a and require the user to manipulate the first band 408a to uncover the port 444a.

In some implementations, as shown in FIG. 10, the wearable device 400′ may also include a wireless magnetic sensor 424 that can be integrated into the device 400′. The wireless magnetic sensor 424 may be positioned on the second surface 402 to magnetically attach to a wireless sensing pad 426. The wireless magnetic sensor 424 may determine whether the wearable device 400′ is magnetically connected to the wireless magnetic pad 426 and removed from the person before allowing the wearable device 400′ to charge. In some implementation, the wireless sensing pad 426 may alone charge the wearable device 400′ either alone and/or simultaneously with a cable.

FIG. 12 illustrates another perspective view of the wearable device 400′ removably connected to a cable C. As illustrated in FIG. 12, the port 444a can be located on a first side 405′ of the plurality of sides 405 such that when a cable C is removably connected to the port 444a, the cable C provides a physical interference that inhibits a first band 408a from wrapping around at least a portion of the user's body. The cable C may be a power and/or data transfer cable that is removably connectable to port 444a. By relocating the port 444a from the position of port 444 shown in FIG. 4C, the user is prevented from charging the battery 133 of device 400′ while the device 400′ is being worn. The user may use and secure the device 400′ after the cable C is detached from the port 444a.

In some implementation, a second band 408b may connect to a second side 405″ opposite of the first side 405′ and at a second band side surface 458. In some implementations, the body portion 416 may include a second connector port 444b positioned opposite of the first connector port 444a on the second side 405″ at a second connector port surface 466 such that when the cable C is removably connected to the port 444b, the cable C provides a physical interference that inhibits a second band 408b of the band 408 from wrapping around at least a portion of the user's body. In some implementations, the second band side surface 458 and the second connector port surface 466 may be substantially vertically aligned with one another. Additionally or alternatively, the second band side surface 458 may partially and/or fully align with the second connector port surface 466. In some implementations, the port 444a and/or port 444b may be closer to the second surface 402 than the first surface 404. For example, as similarly shown in FIG. 10, the port 444b may be a distance D1 from the second surface 402, which is shorter than a distance D2 from the first surface 404. Since D1 is shorter than D2, the connector port 444b may be positioned partially or entirely below the second band 408b. Band 408a thus inhibits connector port 444a from charging when the band 408a is secured around a portion of the user's body. Second band 408b thus inhibits connector port 444b from being charged when the band 408a is secured around a portion of the user's body. In some implementations, the second band side surface 458 and the second connect port surface 466 may be substantially vertically aligned with one another. Additionally or alternatively, the second band side surface 458 may partially and/or fully align with the second connect port surface 466. For example, the second connector port location 466 may be positioned below the second band side surface 458 and between a second band edge 409c and a second band edge 409d of the second band 408b. The port 444b may face in a direction that intersects with the second band 408b such that the second band 408b may cover the port 444b when secured to the user. Additionally or alternatively, the second band 408b may cover the port 444b in a resting position and require the user to manipulate the second band 408b to uncover the port 444b.

In some implementations, the battery 133 disposed in the interior 403 of wearable device 400′ may receive a charge when one or more cables C are simultaneously connected to the first connector port 444a and the second connector port 444b. The dual cable connection may inhibit either band 408a or band 408b or both from wrapping around the user as the device 400′ charges. Additionally or alternatively, the battery 133 in the interior 403 may include a first battery 133a and a second battery 133b in the interior 403. The first battery 133a may be charged when the cable C is removably connected to the port 444a, and the second battery 133b may be charged when an additional cable C is removably connected to the port 444b. The heat source 410 may be operated when both the first battery 133a and the second battery 133b are sufficiently charged to enable the heat source 410 to sufficiently heat a skin surface of the user to at or above 37 degrees Celsius to measure vasodilation.

In some implementations, the port 444a and/or port 444b may be located on the second surface 402. When one or more cables are removably connected to port 444a and/or to port 444b, the one or more cables may provide a physical interference such that band 408a and/or band 408b are prevented from wrapping around at least a portion of the user's body. The second surface 402 may also be prevented from contacting and/or being adjacent to the skin surface of the user. Additionally or alternatively, a third port 444c may be integrated into the device 400′ which may operate in conjunction with port 444a and/or with ports 444a and 444b. The third port may be positioned at a third connector port surface 468 on the second surface 402 such that when a cable C is removably connected to the port 444c, the cable provides a physical interference that inhibits either one or both of band 408a and/or band 408b from wrapping around at least a portion of the user's body. In some implementations, a port positioned on the second surface 402 may include a cover 428 which may deactivate the device 400′ when the cover 428 is in an open configuration such that the port may be removably connected to a cable. Once the cover 428 is moved to a closed configuration, the device 400′ may power on. The cover 428 may also prevent debris, moisture, and the like from entering the port.

### ADDITIONAL CONSIDERATIONS AND TERMINOLOGY

The systems and methods of the implementations described herein and/or variations thereof can be embodied and/or implemented at least in part as a machine may receive a computer-readable medium (or computer program product) storing computer-readable instruction. The instructions are executed by computer-executable components integrated with the system and one or more portions of the hardware processor on the wearable device 102 and/or communicatively coupled computing device. The computer-readable medium (or computer program product) can be stored on any suitable computer-readable media (e.g., memory 118) such as RAMs, ROMs, flash memory, EEPROMs, optical devices (e.g., CD or DVD), hard drives, floppy drives, or any suitable device. The computer-executable component can be a general or application-specific hardware processor, but any suitable dedicated hardware or hardware/firmware combination can alternatively or additionally execute the instructions.

A computer program product can be tangibly embodied in an information carrier. The computer program product may also contain instructions that, when executed, perform one or more methods and/or computer-implemented methods described herein. The information carrier may be a computer- or machine-readable medium, such as the memory 118, or other storage associated with device 102 and/or system 100, system 101, and/or processors 114.

As used in the description and claims, the singular form “a,” “an” and “the” include both singular and plural references unless the context clearly dictates otherwise. For example, the term “signal” may include, and is contemplated to include, a plurality of signals. At times, the claims and disclosure may include terms such as “a plurality,” “one or more,” or “at least one;” however, the absence of such terms is not intended to mean, and should not be interpreted to mean, that a plurality is not conceived.

The term “horizontal” as used herein is defined as a plane parallel to the conventional plane or surface of a heating element (e.g., heat source 410), regardless of its orientation. The term “vertical” refers to a direction perpendicular to the horizontal as just defined. Terms, such as “on,” “above,” “below”, “bottom”, “top”, “side” (as in “sidewall”), “higher”, “lower”, “over”, and “under”, are defined with respect to the horizontal plane.

As used herein, the term “comprising” or “comprises” is intended to mean that the devices, systems, and methods include the recited elements, and may additionally include any other elements. “Consisting essentially of” shall mean that the devices, systems, and methods include the recited elements and exclude other elements of essential significance to the combination for the stated purpose. Thus, a system or method consisting essentially of the elements as defined herein would not exclude other materials, features, or steps that do not materially affect the basic and novel characteristic(s) of the claimed disclosure. “Consisting of” shall mean that the devices, systems, and methods include the recited elements and exclude anything more than a trivial or inconsequential element or step. Implementations defined by each of these transitional terms are within the scope of this disclosure.

Features, materials, characteristics, or groups described in conjunction with a particular aspect, or example are to be understood to be applicable to any other aspect, or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing examples of devices or systems. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that the actual steps taken in the processes illustrated and/or disclosed may differ from those shown in the figures. Depending on the system, certain of the steps described above may be removed, others may be added. Furthermore, the features and attributes of the specific examples disclosed above may be combined in different ways to form additional examples of systems, all of which fall within the scope of the present disclosure. Also, the separation of various system components in the implementations described above should not be understood as requiring such separation in all implementations, and it should be understood that the described components and systems can generally be integrated together in a single product or packaged into multiple products.

Conditional language used herein, such as, among others, “can,” “could,” “might,” “may,” “e.g.,” and the like, unless specifically stated otherwise, or otherwise understood within the context as used, is generally intended to convey that certain features, elements, and/or steps are optional. Thus, such conditional language is not generally intended to imply that features, elements, and/or steps are in any way required or that one or more embodiments necessarily include logic for deciding, with or without other input or prompting, whether these features, elements, and/or steps are included or are to be always performed. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 10 degrees, 5 degrees, 3 degrees, or 1 degree. As another example, in certain embodiments, the terms “generally perpendicular” and “substantially perpendicular” refer to a value, amount, or characteristic that departs from exactly perpendicular by less than or equal to 10 degrees, 5 degrees, 3 degrees, or 1 degree.

The examples and illustrations included herein show, by way of illustration and not of limitation, specific implementations in which the subject matter may be practiced. Other implementations may be utilized and derived therefrom, such that structural and logical substitutions and changes may be made without departing from the scope of this disclosure. Such implementations of the inventive subject matter may be referred to herein individually or collectively by the term “invention” merely for convenience and without intending to voluntarily limit the scope of this application to any single invention or inventive concept, if more than one is in fact disclosed. Thus, although specific implementations have been illustrated and described herein, any arrangement calculated to achieve the same purpose may be substituted for the specific implementations shown. This disclosure is intended to cover any and all adaptations or variations of various implementations. Combinations of the above implementations, and other implementations not specifically described herein, will be apparent to those of skill in the art upon reviewing the above description.

What is claimed is:

1. A temperature safety system for a wearable device, the temperature safety system comprising:
a power source configured to provide power to a heating source;
a first switch configured to receive a first input from a control system; and
a second switch configured to receive a second input from a hardware electronic circuit,
wherein the heating source is configured to receive power from the power source in response to activation of both the first switch and the second switch.

2. The system of claim 1, further comprising a control switch disposed between the power source and the heating source, the control switch configured to receive an input related to an input from at least one of the first switch and the second switch.

3. The system of claim 1, wherein the control system comprises a first temperature sensor configured to measure a first temperature reading, the first temperature reading corresponding to a temperature of a skin surface receiving a stimulus from the heating source.

4. The system of claim 3, wherein the control system comprises one or more processors in communication with the first temperature sensor, the one or more processors configured to receive the first temperature reading from the first temperature sensor.

5. The system of claim 4, wherein the one or more processors are configured to:
receive signals of the first temperature reading from the first temperature sensor;
process the signals of the first temperature reading to determine whether the signals of the first temperature reading are less than a first temperature threshold; and
output a first activation signal or a first cutoff signal at least based on the determination as the first input to the first switch.

6. The system of claim 5, wherein the first temperature threshold is 44° C.

7. The system of claim 6, wherein the first temperature threshold of 44° C. is a maximum temperature threshold.

8. The system of claim 5, wherein the first temperature threshold is 42° C.

9. The system of claim 1, wherein the hardware circuit comprises a second temperature sensor in communication with the heating source, the second temperature sensor configured to measure a second temperature reading, the second temperature reading corresponding to a temperature of the heating source.

10. The system of claim 9, further comprising comparators to compare the second temperature reading to a second threshold value.

11. The system of claim 10, wherein the hardware electronic circuit is configured to:
receive signals of the second temperature reading from the second temperature sensor;
process the signals of the second temperature reading using the comparators to determine whether the signals of the second temperature reading are less than the second temperature threshold; and
output a second activation signal or a second cutoff signal at least based on the determination as the second input to the second switch.

12. The system of claim 1, further comprising one or more control modules in communication with at least one of the first switch and the second switch, the one or more control modules configured to receive an input from at least one of the first switch and the second switch.

13. The system of claim 12, wherein the one or more control modules comprise field effect transistors.

14. A wearable system having a temperature safety system to prevent thermal injury to a person, the system comprising:
a wearable device configured to be in contact with a skin surface of a person, said wearable device comprising:
a stimulus source in communication with the skin surface, wherein the stimulus source is configured to heat the skin surface to a target temperature, wherein the target temperature is a baseline skin temperature of the skin surface plus about 1 degree to about 20 degrees;
a first temperature sensor configured to measure a first temperature reading, the first temperature reading comprising a temperature of the skin surface;
one or more processors in communication with the first temperature sensor, the one or more processors configured to receive the first temperature reading from the first temperature sensor;
a circuit comprising a second temperature sensor in communication with the stimulus source, the second temperature sensor configured to measure a second temperature reading, the second temperature reading comprising a temperature of the stimulus source; and
a power source in electrical communication with the stimulus source and configured to provide power to the stimulus source;
wherein the one or more processors are configured to:
receive signals of the first temperature reading from the first temperature sensor;
process the signals of the first temperature reading to determine whether the signals of the first temperature reading are less than a first temperature threshold; and
output a signal to the power source and the stimulus source to initiate a stimulus cycle, the stimulus cycle comprising heating the skin surface to the target temperature; and
wherein the second temperature sensor is configured to:
receive signals of the second temperature reading from the second temperature sensor;
process the signals of the second temperature reading to determine whether the signals of the second temperature reading are less than a second temperature threshold; and
output a signal to the power source and the stimulus source to initiate a stimulus cycle, the stimulus cycle comprising heating the skin surface to the target temperature.

15. The system of claim 14, wherein the one or more processors are configured to monitor the first temperature reading during the stimulus cycle.
